(19) United States
(12) Patent Application Publication
(10) Pub. No.: US 2010/0280098 A1
(43) Pub. Date: Nov. 4, 2010

(54) RECEPTOR TARGETED OLIGONUCLEOTIDES

(76) Inventors: Rudolph L. Juliano, Chapel Hill, NC (US); Rowshon Alam, Needham, MA (US); Vidula Dixit, Madison, AL (US); Hyunmin Kang, Pomona, CA (US); Xiaoyuan Chen, Potomac, MD (US); Zi-Bo Li, Logan, UT (US)

Correspondence Address: JENKINS, WILSON, TAYLOR & HUNT, P. A., 3100 Tower Blvd., Suite 1200, DURHAM, NC, 27707, US

(21) Appl. No.: 12/681,260

(22) PCT Filed: Oct. 6, 2008
(86) PCT No.: PCT/US08/11511

§ 371 Date: Jul. 14, 2010

Related U.S. Application Data
(60) Provisional application No. 60/998,027, filed on Oct. 5, 2007.

(51) Int. Cl. A61K 31/7105; C12N 5/071; A61P 43/00

(52) U.S. Cl. 514/44 A; 435/325

(57) ABSTRACT

Disclosed herein are oligonucleotide conjugates that include ligands that target cell receptors that mediate endocytosis. The ligands can include peptides and small molecules. The conjugates can include carrier macromolecules to which the ligands and oligonucleotides are attached, or conjugates where an oligonucleotide is attached to a ligand in the absence of a carrier macromolecule. The oligonucleotides can include therapeutic oligonucleotides, such as siRNA, antisense RNA and miRNA. The ligand can be an RGD peptide. Also disclosed herein are methods of delivering the conjugates to cells in subjects.

### RELATED APPLICATIONS

This application claims the benefit of U.S. Provisional Patent Application Ser. No. 60/998,027, filed Oct. 5, 2007; the disclosure of which is incorporated herein by reference in its entirety.

### GOVERNMENT INTEREST

This presently disclosed subject matter was made with U.S. Government support under Grant No. PO1 GM59299 awarded by the National Institutes of Health and Grant Nos. R21 CA121842, P50 CA114747, and U54CA119367 awarded by the National Cancer Institute and Grant No. W81XWH-07-1-0374 awarded by the Department of Defense. Thus, the U.S. Government has certain rights in the presently disclosed subject matter.

### TECHNICAL FIELD

The presently disclosed subject matter pertains at least in part to therapeutic delivery of antisense, siRNA and miRNA oligonucleotides. The oligonucleotides can be delivered as conjugates comprising ligands that target cell receptors that mediate endocytosis.

### ABBREVIATIONS

° C.=degrees Celsius
Å=angstrom
μg=micrograms
CBQCA=(3-(4-carboxybenzoyl)quinoline-2-carboxaldehyde
CH₃CN=acetonitrile
CPA=cysteine-PEG-albumin conjugate
CPG=controlled pore glass
CPP=cell penetrating peptides
Cys=cysteine
DIC=differential interference contrast
DMEM=Dulbecco's minimum essential medium
DTT=dithiothreitol
EDTA=ethylenediamine tetraacetate
FBS=fetal bovine serum
FPLC=fast protein liquid chromatography
h=hours
HEPES=(4-(2-hydroxyethyl)-1-piperazine-ethane sulfoninc acid
HPLC=high performance liquid chromatography
HSA=human serum albumin
kDa=kilodaltons
M=molar
Mal=maleimide
MALDI-TOF=matrix-assisted laser assisted/desorption time-of-flight
Me=methyl
mg=milligrams
min=minutes
miRNA=micro-ribonucleic acid
mL=milliliter
mM=millimolar
mPEG=methoxy poly(ethylene glycol)
MS=mass spectroscopy
MW=molecular weight
MW_{calcd}=molecular weight calculated
MW_{found}=molecular weight found
NHS=N-hydroxysuccinimide
nm=nanometer
nM=nanomolar
PA=pegylated albumin
PBS=phosphate buffered saline
PEG=poly(ethylene glycol)
PEO=polyethylene oxide
PNA=peptide nucleic acid
QELS=quasi-elastic dynamic light scattering
RFUs=relative fluorescence units
RLUs=relative luciferase units
RNA=ribonucleic acid
RPA=RGD_PEG-albumin conjugate
RPAO═RGD-PEG-albumin-oligo-nucleotide conjugate
RT=room temperature
s=seconds
SDS-PAGE=sodium dodecyl sulfate polyacrylamide gel electrophoresis
sRNA=small interfering ribonucleic acid
SSO=splice shifting oligonucleotide
TEAA=triethylammonium acetate
UV=ultraviolet

### BACKGROUND

Antisense oligonucleotides, small interfering RNAs (sRNA), and micro RNAs (miRNA) have elicited great interest both as laboratory reagents and as possible therapeutic entities. There have been attempts to address a number of issues associated with the use of oligonucleotides, including potency and stability in the biological milieu, through a variety of chemical modifications. See Kurreck, (2003)Eur. J. Biochem., 270, 1628-1644; Manoharan (2002) Antisense Nucleic Acid Drug Dev., 12, 103-128; and Fisher et al. (2007) Nucleic Acids Res., 35, 1064-1074. However, in terms of therapeutic use of antisense, sRNA or miRNA oligonucleotides, a key remaining issue is that of effective delivery. See Juliano and Yoo (2000) Curr. Opin. Mol. Ther., 2, 297-303; and Inoue et al. (2006) J. Drug Target., 14, 448-455. There is abundant evidence that both antisense and siRNA oligonucleotides can exert therapeutic effects in animal models when administered as ‘free’ compounds, that is, in the absence of any delivery agent. See Crooke (2004) Annu. Rev. Med., 55, 61-95; Soutschek et al. (2004) Nature, 432, 173-178; and Kim and Rossi (2007) Nat. Rev. Genet., 8, 173-184. Moreover, a number of clinical trials of both antisense and siRNA agents as free compounds are underway. See Coppelli and Grandis (2005) Curr. Pharm. Des., 11, 2825-2840. Nonetheless, the possible improvement of the therapeutic potential of oligonucleotides by the use of appropriate delivery systems continues to represent a perceived need in the art.

A variety of approaches have been attempted to enhance delivery of siRNA and antisense oligonucleotides. In the case of siRNA, viral vector systems are a possibility. See Van den Haute et al. (2003) Hum. Gene Ther., 14, 1799-1807; McCaffrey et al., (2002) Nature, 418, 38-39; and Xu et al. (2005) Mol. Ther., 11, 523-530. Various synthetic nanocarriers including liposomes, polymeric nanoparticles and dendrimers have also been extensively studied as oligonucleotide delivery agents. See Hu-Lieskovan et al. (2005) Cancer Research, 65, 8984-8992; Oishi et al. (2005) Biomacromolecules, 6, 2449-2454; Vinogradov et al. (2004) Bioconjug. Chem., 15, 50-60; Fattal et al. (2004) Adv. Drug Deliv. Rev., 56, 931-946; and Yoo and Juliano (2000) Nucleic Acids Res. 28, 4225-4231. However, the particulate nature of these materials can limit their biodistribution in vivo. See Juliano (2007) in Mirkin and Niemeyer (eds.), Nanobiotechnology, Wiley and Sons, New York, Vol. 2. Another attempted approach has been to chemically conjugate oligonucleotides with various peptide ligands, including so called ‘cell penetrating peptides’ (CPPs). A number of CPPs have been described; they are most commonly polycationic sequences that seem to have the ability to penetrate from the outside of the cell to the cytosol, and in doing so to assist in delivery of linked ‘cargo’ molecules, including peptides and proteins. See Jarver and Lanoel (2004) Drug Discov. Today, 9, 395-402; and Wadia and Dowdy (2002) Curr. Opin. Biotechnol. 13, 52-56. A considerable effort has gone into the preparation and evaluation of conjugates of CPPs and oligonucleotides; however, on the whole this has been only modestly successful. See Juliano (2005) Curr. Opin. Mol. Ther., 7, 132-136; and Abes et al. (2007) Biochem. Soc. Trans., 35, 53-55. While some biological effect from conjugates of CPPs with anionic antisense oligonucleotides (see Astriab-Fisher et al. (2002) Pharm. Res., 19, 744-754; and Astriab-Fisher et al. (2000) Biochem. Pharmacol., 60, 83-90) and from CPP-siRNA conjugates has been reported (see Muratovska and Eccles (2004) FEBS Lett., 558, 63-68; and Chiu et al. (2004) Chem. Biol., 11, 1165-1175), the prevailing view in the art suggests that, at best, CPPs are only able to effectively deliver oligonucleotides with uncharged backbones, such as peptide nucleic acids (PNA) and morpholino compounds. See Turner et al. (2005) Nucleic Acids Res., 33, 6837-6849; El-Andaloussi et al. (2006) The Journal of Gene Medicine, 8, 1262-1273; Bendifallah et al. (2006) Bioconjug. Chem., 17, 750-758; Moulton et al. (2003) Antisense Nucleic Acid Drug Dev., 13, 31-43; and Abes et al. (2007) Nucleic Acids Res., 35, 4495-4502.

Accordingly, there exists a need for additional methods and compositions for delivering therapeutic oligonucleotides to cells with high efficiency and low toxicity, regardless of backbone polarity.

### SUMMARY

In some embodiments, the presently disclosed subject matter provides a composition for delivering an oligonucleotide to a target cell through endocytosis, the composition comprising one or more ligand groups capable of mediating receptor endocytosis and one or more oligonucleotide groups each comprising an oligonucleotide, wherein the composition comprises a conjugate comprising a ligand group attached to an oligonucleotide group or a conjugate comprising one or more ligand groups, one or more oligonucleotide groups, and a carrier macromolecule, wherein each of the ligand groups and each of the oligonucleotide groups are attached to the carrier macromolecule.

In some embodiments, the oligonucleotide comprises one of the group consisting of an antisense RNA, a small interfering RNA (siRNA), and a micro RNA (miRNA) that selectively binds to an RNA in the target cell. In some embodiments, the one or more oligonucleotide groups further comprise a detectable tag. In some embodiments, the detectable tag is attached to a 3′ end of the oligonucleotide. In some embodiments, the detectable tag is a fluorophore. In some embodiments, the detectable tag is a Tamra fluor.

In some embodiments, the oligonucleotide is a therapeutic agent and the composition comprises a therapeutically effective amount of the oligonucleotide. In some embodiments, the composition is prepared for administration to a vertebrate subject. In some embodiments, the composition is prepared as a pharmaceutical formulation for administration to a mammalian subject.

In some embodiments, the one or more ligand groups each comprise one or more peptide ligand. In some embodiments, the peptide ligand is a cyclic RGD peptide. In some embodiments, one or more ligand group comprises a combination of different types of ligands. In some embodiments, the different types of ligands are selected from the group consisting of EGF, CXCL12, CCL3, small organic molecule ligands for chemokine receptors, small organic molecule ligands for the P2Y subfamily of GPCR receptors, small organic molecule ligands for alpha and beta adrenergic receptors, terbutaline, phenylephrine, and peptide, peptidomimetic and non-peptide ligands for integrins including a5b1, a4b1 and LFA-1.

In some embodiments, the compostion comprises a conjugate comprising one or more ligand groups and the one or more oligonucleotide groups each attached to a carrier macromolecule. In some embodiments, the carrier macromolecule is a protein. In some embodiments, the carrier macromolecule is a serum albumin protein. In some embodiments, the carrier macromolecule is human serum albumin.

In some embodiments, one or more ligand group comprises a polyethylene glycol (PEG) moiety, wherein the PEG moiety is attached to the protein. In some embodiments, the PEG moiety is attached to the protein through an amide linkage. In some embodiments, one or more ligand group comprises a cyclic RGD peptide attached to the PEG group through a maleimide group.

In some embodiments, the one or more oligonucleotide groups are attached to the carrier macromolecule through an alkylene linker group. In some embodiments, the alkylene linker group is —S—(CH₂)₆—.

In some embodiments, the composition comprises a ligand group attached conjugated to an oligonucleotide group. In some embodiments, the peptide ligand is a multivalent peptide ligand. In some embodiments, the multivalent peptide ligand is selected from the group consisting of a bi-, tri-, tetra-, penta-, hexa-, and octa-valent peptide ligand. In some embodiments, the multivalent peptide ligand is a bicyclic RGD peptide. In some embodiments, the bicyclic RGD peptide is linked to a maleimide group and the maleimide group is linked to the oligonucleotide group through an alkylene linker group. In some embodiments, the alkylene linker group is —S—(CH₂)₆—.

In some embodiments, the presently disclosed subject matter provides a method of delivering an oligonucleotide to a target cell through endocytosis, wherein the method comprises contacting the cell with a composition comprising one or more ligand groups capable of mediating receptor endocytosis and one or more oligonucleotide groups each comprising an oligonucleotide, wherein the oligonucleotide is actively transported into the target cell, wherein the target cell comprises one or more receptors capable of mediating receptor endocytosis in response to the one or more ligand groups, and wherein the composition comprises a conjugate comprising a ligand group attached to an oligonucleotide group or a conjugate comprising one or more ligand groups, one or more oligonucleotide groups, and a carrier macromolecule, wherein each of the ligand groups and each of the oligonucleotide groups are attached to the carrier macromolecule. In some embodiments, the target cell is present in a subject, and contacting the target cell with the composition comprises administering a therapeutically effective amount of the composition to the subject.

Accordingly, it is an object of the presently disclosed subject matter to provide compositions and methods for delivering oligonucleotides to target cells via receptor mediated endocytosis.

An object of the presently disclosed subject matter having been stated hereinabove, which is addressed in whole or in part by the presently disclosed subject matter, other objects and aspects will become evident as the description proceeds when taken in connection with the accompanying description, Figures and Examples as best described herein below.

### BRIEF DESCRIPTION OF THE DRAWINGS

FIG. 1A is a schematic illustration of the chemical structure of a maleimide-bicyclic RGD peptide. The maleimide reactive group is positioned mid-way along a linker that joins the two cyclic RGD moieties.

FIG. 1B is a scheme for the conjugation of an antisense oligonucleotide group based on oligonucleotide 623 (SEQ ID NO: 1) to a bivalent RGD peptide. Reagent conditions (I) are 100 mM dithiothreitol (DTT), 0.1M triethylammonium acetate (TEAA) buffer, and 1% triethylamine. Reagent conditions (II) are maleimide-bicyclic-RGD peptide in H₂O (1.5 equivalent), 400 mM potassium chloride (KCl), 40% acetonitrile (CH₃CN) for three hours at room temperature. FIG. 1C is a graph showing high performance liquid chromatography (HPLC) analysis of the RGD peptide-oligonucleotide conjugate. The elution profiles of the 5′-SH-3′-Tamra 623 oligonucleotide (dotted line) and its bivalent RGD conjugate (solid line) are shown.

FIG. 2A is a graph showing the results of dose-response studies in cells treated with either 623-Tamra, RDG-623-Tamra conjugate, or 623-Tamra complexed with LIPOFECTAMINE™ 2000. Luciferase activity was determined after 48 h and expressed as relative luciferase units (RLUs) per 1.5×10⁵ cells. Black bars represent luciferase activity in 623-Tamra-treated cells, striped bars represent luciferase activity in RDG-623-Tamra conjugate-treated cells, and grey bars represent luciferase activity in cells treated with 100 nM 623-Tamra transfected using LIPOFECTAMINE™ 2000. Results are the means and standard errors of three determinations.

FIG. 2B is a graph showing the effect of 623-Tamra or RGD-623-Tamra compared to controls based on an antisense oligonucleotide having 5 mismatched bases (indicated as 5MM623 (SEQ ID NO: 2)). The conjugates or free oligonucleotide were used at 200 nM while the LIPOFECTAMINE™ 2000 complexes were used at 100 nM oligonucleotide. Results are the means and standard errors of three determinations.

FIG. 3 is a graph showing the results of time-response studies. Cells were treated with either 623-Tamra, RDG-623-Tamra conjugate, or 623-Tamra complexed with LIPOFECTAMINE™ 2000, and luciferase activity was determined at 24 hours, 48 hours, 72 hours, 96 hours, or 120 hours, as indicated on the x axis. Black bars represent luciferase activity in cells treated with 200 nM 623-Tamra, striped bars represent activity in cells treated with 200 nM RDG-623-Tamra conjugate, and grey bars represent activity in cells treated with 100 nM 623-Tamra complexed to LIPOFECTAMINE™ 2000, all expressed as relative luciferase units (RLUs) per 10⁵ cells. Results are the means and standard errors of triplicate determinations.

FIG. 4A is a graph showing the results of total cellular uptake studies. Cells were treated with 12.5, 50, or 100 nM of either 623-Tamra (shaded bars) or RGD-623-Tamra conjugate (striped bars) for four hours. Results represent means and standard deviations of triplicate determinations and are expressed as relative fluorescence units (RFUs) per microgram cell protein.

FIG. 4B is a bar graph of 623-Tamra (shaded bars) and RGD-623-Tamra conjugate (striped bars) uptake in αvβ3-positive M21+ human melanoma cells (left side of graph) and αvβ3-negative M21− human melanoma cells (right side of graph). The cells were exposed to 12.5, 25, 50, or 100 nM of either 623-Tamra or RGD-623-Tamra conjugate. Results represent means and standard errors of triplicate determinations and are expressed as RFUs per microgram cell protein.

FIG. 4C are a series of 4 panels of flow cytometry analyses together showing the lack of down-regulation of αvβ3 by RGD-623 conjugate. A375SM-Luc705-B cells were either maintained as controls or treated with 200 nM RGD-623 Tamra conjugate or 623-Tamra for 24 h. Then, αvβ3 levels were determined by immunostaining with anti-αvβ3 antibody. Panel (1) in the upper left shows cells not stained with primary anti-αvβ3. Panel (2) in the upper right shows control cells stained with anti-αvβ3. Panel (3) in the lower left shows cells treated with 200 nM of RGD-623-Tamra and stained with anti-αvβ3. Panel (4) in the lower right shows cells treated with 200 nM of 623-Tamra and stained with anti-αvβ3. The y axis of each panel indicates the number of cells, while the x axis indicates the log of fluorescence intensity.

FIG. 5 is a bar graph showing the inhibitory effect of excess RGD peptide. Free RGDfV peptide was added to cells at a concentration of 0.1, 1, 10, or 100 μM 30 minutes prior to treatment with either 623-Tamra or RGD-623-Tamra conjugate. Luciferase activity was determined after 48 hours. The dotted line represents luciferase activity of 200 nM 623-Tamra, and the solid line represents activity of 200 nM RGD-623-Tamra conjugate. Results are the means and standard errors of triplicate determinations.

FIG. 6A is a series of micrographs showing the subcellular distribution of 200 nM RGD-623-Tamra conjugate (Panel (1) on the left), 200 nM 623-Tamra (Panel 2 in the center), and 623-Tamra complexed with LIPOFECTAMINE™ 2000 (100 nM; Panel 3 on the right) in A375SM-Luc705-B cells. Tamra-related fluorescence was observed in cells in panels (1) and (3). In panel (1) the white arrows indicate the position of the nucleus, which were generally free of fluorescence, while in panel (3), black arrows indicate nuclei that display fluorescence related to having accumulated Tamra-oligonucleotide. Very little fluorescence related to cellular uptake of Tamra was observed in the cells in panel 2.

FIG. 6B is a series of micrographs showing the co-localization of endosomal pathway markers with RGD-623-Tamra. The pair of panels (1) in the upper left show co-localization in cells treated with RGD-623-Tamra conjugate (100 nM) co-incubated with Transferrin-Alexa 488 (200 nM) for 2 h. The pair of panels (2) in the upper right show co-localization in cells treated with RGD-623-Tamra conjugate (100 nM) co-incubated with Transferrin-Alexa 488 (200 nM) for 24 h. The pair of panels (3) in the lower left show co-localization in cells treated with RGD-623-Tamra conjugate (100 nM) co-incubated with Dextrin-Alexa 488 (2 μM) for 2 h. The pair of panels (4) in the lower right show co-localization in cells treated with RGD-623-Tamra conjugate (100 nM) co-incubated with Dextrin-Alexa 488 (2 μM) for 24 h. The right panel of panels (1) shows no overlap of RGD-Tamra fluorescence and Transferrin-Alexa 488 fluorescence. Substantial overlap was seen in the right panels of panel pairs (2), (3), and (4).

FIG. 7A is a series of micrographs showing the co-localization of caveolin-1 and RGD-623-Tamra. Cells were treated with 50 nM RGD-623-Tamra conjugate for 6 hours then fixed, permeabilized and stained with antibodies of sub-cellular compartments, followed by Alexa 488 secondary antibody. The panel on the left shows sub-cellular localization of caveolin-1. The panel in the center shows the sub-cellular localization of RGD-623-Tamra conjugate. The panel on the right shows the sub-cellular localization of both RGD-623-Tamra conjugate and caveolin-1. Fletches and boxes indicate areas of co-localization. Boxed areas are shown at higher magnification.

FIG. 7B is a series of micrographs showing the co-localization of αvβ3 and RGD-623-Tamra. Cells were treated with 50 nM RGD-623-Tamra conjugate for 6 hours then fixed, permeabilized and stained with antibodies of sub-cellular compartments, followed by Alexa 488 secondary antibody. The panel on the left shows sub-cellular localization of αvβ3. The panel in the center shows the sub-cellular localization of RGD-623-Tamra conjugate. The panel on the right shows the sub-cellular localization of both RGD-623-Tamra conjugate and αvβ3. Fletches and boxes indicate areas of co-localization. Boxed areas are shown at higher magnification.

FIG. 7C is a series of micrographs showing the co-localization of trans-Golgi marker TGN230 and RGD-623-Tamra. Cells were treated with 50 nM RGD-623-Tamra conjugate for 6 hours (upper panels) or 24 hours (lower panels), then fixed, permeabilized and stained with antibodies of sub-cellular compartments, followed by Alexa 488 secondary antibody. Panels on the left show sub-cellular localization of trans-Golgi marker TGN230. Panels in the center show the sub-cellular localization of RGD-623-Tamra conjugate. Panels on the right show the sub-cellular localization of both RGD-623-Tamra conjugate and trans-Golgi marker TGN230. Fletches and boxes indicate areas of co-localization. Boxed areas are shown at higher magnification.

FIG. 7D is a bar graph of cellular uptake of RGD-623-Tamra in cells treated with cytochalasin D (darkly shaded bars) or β-cyclodextrin (striped bars). Cells were treated with 1 mM, 5 mM, or 10 mM β-cyclodextrin or 0.2 μM, 2 μM or 20 μM cytochalasin D as indictaed on the x-axis for 15 minutes, and then 100 nM RGD-623-Tamra was added. Total cell uptake after 4 h was measured. Uptake in control cells (lightly shaded bar) that had not been treated with either 3-cyclodextrin or cytochalasin D is also shown. No loss of cell viability was detected at the concentrations used, although the highest concentration of cytochalasin D caused some cell rounding. Results represent means and standard error of triplicate determinations and are normalized based on cells receiving no inhibitor as 100%.

FIG. 8 is a bar graph showing the short-term toxicity of 623-RGD conjugates. Cells were treated with either 623-Tamra (darkly shaded bars), 623-Tamra complexed with LIPOFECTAMINE™ 2000 (lightly shaded bar, third from left) or RGD-623-Tamra conjugate (striped bars). Concentrations of 623-Tamra and RGD-623-Tamra conjugate were 50, 250, 500, or 1000 nM as indicated on the x-axis. Controls also include cells treated with LIPOFECTAMINE™ 2000 alone (lightly shaded bar, second from left) and untreated cells (lightly shaded bar, first on left). Results are means and standard errors of three determinations.

FIG. 9A is a schematic showing the preparation of cleavable oligonucleotide conjugates of human serum albumin with cRGD peptide. Alexa 488-Mal=Alexa Fluor 488 C5 maleimide; MaI-PEG-NHS=Malhex-NH-PEG-O—C3H6-CONHS; cRGD-SH=cyclo[RGDfK-COCH₂SH]; SPDP=Sulfo-LC-SPDP (i.e., Sulfosuccinimidyl-6-(3′-(2-pyridyldithio)-propionamido-hexanoate); Oligo-SH=623-SH (the thiol derivative of SEQ ID NO: 1), 5MM623-SH (the thiol derivative of SEQ ID NO: 2); or Tamra-5MM623-SH (the tagged and thiolated derivative of SEQ ID NO: 2). A is human serum albumin (HSA); PA is PEG-HSA conjugate; RPA is RGD-PEG-HSA conjugate; and RPAO is RGD-PEG-HSA-oligonucleotide conjugate.

FIG. 9B is a schematic showing the chemistry of the maleimide-PEG NHS ester moiety of the oligonucleotide conjugate of FIG. 9A.

FIG. 10 are a series of UV spectra showing the cleavable disulfide formation between RGD-HSA conjugate and thiolated oligonucleotide. Spectra (A) on the upper left is the UV spectra of RGD-HSA conjugate (RPA). Spectra (B) on the right is the UV spectra of a reaction mixture of 623-SH and the RGD-HSA-SPDP conjugate formed from the reaction of RGD-HSA and sulfo-LC-SPDP. Spectra (C) on the lower left is the UV spectra of the RGD-HSA-623 conjugate. In spectras (A), (B), and (C), arrows marked 1 indicate the peak for Alexa 488, arrows marked 2 indicated the peak for pyridine-2-thione, and the arrows marked 3 indicate the peak for oligonucleotide.

FIG. 11 are photographs showing the analysis of oligonucleotide conjugates (photograph A on the left) and nuclease resistance (photograph B on the right) by polyacrylamide gel electrophoresis. In photograph (A): Lane 1 is HSA-Alexa 488; Lane 2 is PEG-HSA conjugate (PA); Lane 3 is RGD-PEG-HSA conjugate (RPA); Lane 4 is 623-Tamra; and Lane 5 is RGD-PEG-HSA-623-Tamra conjugate. In photograph (B), lanes 1-5 are RGD-PEG-HSA-623-Tamra conjugate digested with Micrococcal nuclease (400 gel units) for 0 h, 1 h, 2 h, 4 h, and 12 h, respectively. Lanes 6-10 are 623-Tamra digested with Micrococcal nuclease (400 gel units) for 0 h, 1 h, 2 h, 4 h, and 12 h, respectively.

FIG. 12 are spectra showing the fast protein liquid chromatograph (FPLC)/quasi-elastic dynamic light scattering (QELS) analysis of human serum albumin (HSA; upper spectra); 623-SH oligonucleotide (middle spectra), and RGD-PEG-HSA-623 conjugate (lower spectra), indicating molecular size and polydispersity. Faster migrating shoulder peaks seen in the HSA and 623-SH samples are believed to represent S—S bridged dimers.

FIG. 13 is a bar graph of dose response and specificity studies with RGD-HSA-oligonucleotide conjugates. Cells were treated with free 623 (SEQ ID NO: 1; lightly shaded bars), RGD-PEG-HSA-MM (the peptide-HSA-oligonucleotide conjugate with SEQ ID NO: 2; bars with vertical stripes), a cysteine-PEG-HSA conjugate prepared conjugating cysteine with the maleimide on PEG; bars with horizontal stripes); RGD-PEG-HSA-623 conjugate (RPA-623; darkly shaded bars) at 25, 50, 100, and 200 nM, or LIPOFECTAMINE™ 200 complexed to 623-SH (unshaded bars) at either 100 nM or 200 nM. Luciferase activity was determined after 72 h from the cell lysates and expressed as relative luminescence units (RLUs) per μg of protein. Results are means and standard error of three determinations.

FIG. 14 is a bar graph showing the results of time response studies with RGD-HSA-623 conjugates. Cells were treated with 200 nM of free 623 (SEQ ID NO: 1; lightly shaded bars), RGD-PEG-HSA-623 (RPA-623; darkly shaded bars) of 623 (SEQ ID NO: 1) complexed to LIPOFECTAMINE™ 2000 (L2/623, 1.5 μg/mL, unshaded bars). Luciferase activity was determined from cell lysates collected at various times and expressed as relative luminescence units (RLUs) per microgram of cell protein. Results are means and standard errors of three determinations.

FIG. 15 is a bar graph showing oligonucleotide antisense effect by excess cRGD peptide. Free cyclo RGDfV peptide was added at 0, 0.1, 1, and 10 μM to cells 30 min prior to treatment with either free 623 (SEQ ID NO: 1; 100 nM, lighter shaded bars) or RGD-PEG-HSA-623 conjugate (100 nM, darker shaded bars). Luciferase activity was determined after 48 h from cell lysates and expressed as relative luminescence units (RLUs) per microgram of cell protein. Results are means and standard errors of three determinations.

FIG. 16 is a graph showing the cellular accumulation of fluorescence in cells treated with 100 nM of free 623-Tamra (open squares), 623-Tamra complexed with LIPOFECTAMINE™ 2000 (1.5 μg/mL; open diamonds), RGD-PEG-HSA-MM oligonucleotide conjugate (dark circles), RGD-PEG-HSA-623-Tamra conjugate (dark squares), or cysteine-PEG-HSA-623-Tamra conjugate (open circles) after 2, 4, 6, 8, and 10 h. After incubation, cells were washed and lysates were analyzed using a fluorimeter for uptake measurements. Results are means and standard errors of three determinations.

FIG. 17A is a pair of panels showing the confocal microscopy analysis of cellular uptake of 100 nM of free 623-Tamra. No Tamra fluorophore appeared to have accumulated in nuclei.

FIG. 17B is a pair of panels showing the confocal microscopy analysis of cellular uptake of 100 nM of 623-Tamra complexed with LIPOFECTAMINE™ 2000 (1.5 μg/mL). Arrows indicated Tamra fluorophore accumulated in nuclei.

FIG. 17C is a pair of panels showing the confocal microscopy analysis of cellular uptake of 100 nM of cysteine-PEG-HSA-623-Tamra conjugate. No Tamra fluorophore appeared to have accumulated in nuclei.

FIG. 17D is a pair of panels showing the confocal microscopy analysis of cellular uptake of 100 nM of RGD-PEG-HSA-623-Tamra conjugate. Arrows indicated Tamra fluorophore accumulated in nuclei.

FIG. 18 are a series of images of co-localization studies of 623 (SEQ ID NO: 1) with endosomal pathway markers. An RGD-PEG-HSA-623-Tamra conjugate (RPA-623-Tamra; 100 nM) prepared by conjugtating an HSA surface thiol group to a cysteine instead of Alexa 488 was coincubated with (row A) Transferrin-Alexa 488 (200 nM) for 2 h, (row B) Transferrin-Alexa 488 (100 nM) for 24 h, (row C) Dextran-Alexa-488 (2 μM) for 2 h, and (row D) Dextran-Alexa-488 (2 μM) for 24 h. Live cells were observed for (column 1) Alexa-488 and (column 2) Tamra. Column (3) are the merged images for Alexa-488 and Tamra and column (4) are differential interference contrast (DIC) images. Arrows indicate co-localization of Alexa 488 and Tamra fluorophores.

FIG. 19 is a bar graph of cellular uptake inhibition studies with RGD-HSA-oligonucleotide conjugates. Cells were treated with β-cyclodextrin, cytochalasin D, or cyclo RGDfV (cRGD) for 30 min prior to treatment with either free 623-Tamra (100 nM) control (bar on left) or RGD-PEG-HSA-623-Tamra (RPA-623-Tamra, 100 nM). After 4 h, the cells were washed and lysates were analyzed for uptake. Results are expressed as relative fluorescence units (RFUs), percentages of the fluorescence of the control and are means and standard errors of three determinations.

FIG. 20 is a graph of toxicity studies with RGD-HSA-oligonucleotide conjugates. Cells were treated with free 623 (SEQ ID NO: 1), LIPOFECTAMINE™ 2000/623 complex (L2/623), or RGD-PEG-HSA-623 conjugate (RPA-623). After 48 hours, cells were trypanized and viable cells were counted for short-term studies (shown as bars, see also (A) on left y-axis). Alternatively, cells were re-plated in 6 well plates containing a mixture of 1% low gelling temperature agarose and complete DMEM medium with 10% FBS for long-term toxicity studies (shown in circles and line; see also (B) on right axis). After 14 days, surviving colonies larger than 25 cells were counted. Survival is expressed as colonies per 100 cells plated.

FIG. 21 are photographs of intracellular uptake of oligonucleotides in tumor-bearing mice. A375 melanoma cells containing an inducible luciferase reporter gene were use as xenografts in SCID mice and placed on the right flank. Mice were injected with (1) saline, (2) RGD-PEG-HSA-623, or (3) free 623 (SEQ ID NO: 1). Mice were later injected with luciferin and the luciferase activity in the tumors was monitored by bioluminescence imaging. The arrow indicates a tumor where substantial increase in luciferase activity was detected.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

### I. Definitions

Following long-standing patent law convention, the terms “a”, “an”, and “the” refer to “one or more” when used in this application, including the claims. Thus, for example, reference to “a conjugated cyclic RGD-terminated group” includes one or more conjugated cyclic RGD-terminated groups, two or more conjugated cyclic RGD-terminated groups, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical region of parameters set forth in this specification and attached claims are approximations that can vary depending upon the desired properties sought to be obtained by the presently disclosed subject matter.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl)hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, methylpropynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described.

In some embodiments, the compounds and conjugates described by the presently disclosed subject matter contain a linking group. As used herein, the term “linking group” comprises a chemical moiety, such as an alkylene, arylene (such as a furanyl, phenylene, thienyl, and pyrrolyl radical), or other group which can be bonded to two or more other chemical moieties to link the moieties.

The term “macromolecule” as used herein generally refers to synthetic organic or inorganic polymers and to biological polymers (e.g., proteins). Typically, macromolecules are molecules having a molecular weight (MW) of 1000 Daltons or more. A “carrier macromolecule” is a macromolecule that can be conjugated to one or more targeting, therapeutic, or detection moiety, and which does not, by itself, have any therapeutic or toxic effect. In some embodiments, the carrier macromolecules can be used to increase the MW of therapeutic and targeting groups to slow down their elimination from a biological system.

The terms “targeting group” or “ligand” as used herein refer to a moiety that binds such as but not limited to through a receptor on a target cell. In some embodiments, the ligand binds to a receptor capable of mediating receptor endocytosis. In some embodiments, the ligand is a peptide, a small molecule or combinations thereof. Ligands can be “multivalent” (i.e., one ligand can bind to two or more receptors at the same time). For example, typically, one portion of the ligand interacts with the binding pocket of the receptor. In the case of multivalent ligands, the ligand can include two or more copies of the portion that interacts with the binding pocket of the receptor.

As used herein, the term “peptide” means any polymer comprising any of the 20 protein amino acids or any non-naturally occurring amino acid, regardless of its size. Although “protein” is often used in reference to relatively large polypeptides (e.g., polypeptides comprising more than about 100, 200, 300, 400, or 500 amino acids) and “peptide” is often used in reference to small polypeptides, usage of these terms in the art overlaps and varies. The term “polypeptide” as used herein refers to peptides, polypeptides and proteins, unless otherwise noted.

As used herein, the terms “nucleic acid”, “oligonucleotide” and “polynucleotide” are used interchangeably and refer to a deoxyribonucleotide or ribonucleotide polymer in either single- or double-stranded form, and unless otherwise limited, encompass known analogues of natural nucleotides that hybridize to nucleic acids in manner similar to naturally occurring nucleotides. An oligonucleotide can be chemically synthesized, excised from a larger polynucleotide or can be isolated from a host cell or organism. A particular polynucleotide can contain both naturally occurring residues as well as synthetic residues. Unless otherwise indicated, a particular nucleic acid sequence includes the complementary sequence thereof. In some embodiments, the term oligonucleotide can be used to refer to a polynucleotide that is 50 nucleotides long or less.

The term “antisense oligonucleotide” refers to a single stranded RNA that comprises a complementary sequence to a messenger RNA (mRNA) and that can inhibit translation of the mRNA, thereby interferring with expression of a gene.

The term “siRNA” refers to a double stranded RNA, with or without overhangs, that can interfere with the expression of a gene. The siRNA can be less than 50 nucleotides long. In some embodiments, the siRNA can be between about 15 and about 35 nucleotides long. In some embodiments, the siRNA is between about 20 and 25 nucloetides long.

The term “miRNA” refers to a single stranded RNA that can regulate gene expression. The miRNA can be between about 15 and about 50 nucleotides long. Typically, miRNAs are between about 21 and 25 nucleotides long.

The use of the term “free” when used in conjunction with any oligonucleotide refers to an oligonucleotide or oligonucleotide derivative that is free of a targeting moiety and/or a carrier macromolecule.

As used herein, the terms “RGD peptide” and “RGD” refer to peptides or polypeptide-containing molecules having at least one arginine (Arg)-glycine (Gly)-aspartic acid (Asp) sequence (SEQ ID NO: 3) or a functional equivalent.

As used herein, the term “polyethylene glycol” (i.e., PEG) is meant to refer to common derivatives of PEG and polyethylene oxide (PEO). For example, the term PEG includes the use of methyl ether (methoxypoly (ethylene glycol), (i.e., mPEG). PEG and PEO melting points can vary depending on the formula weight of the polymer. PEG or PEO can have the following structure: HO—(CH₂—CH₂—O)ₙ—H.

The term “detectable tag” as used herein refers to a signal-producing tag (e.g., an enzyme, fluorophore, luminophore, radioisotope, etc.) which is capable of detection either directly or through its interaction with a substance such as a substrate (in the case of an enzyme), a light source (in the case of a fluorescent compound), or a photomultiplier tube (in the case of a radioactive or chemiluminescent compound). In some embodiments, the detectable tag is a fluorescent tag. Fluorescent tags are moieties that, after absorption of energy, emit radiation at a defined wavelength. Many suitable fluorescent tags that can be incorporated or attached to nucleic acid sequences are known. Fluorescent tags that can be utilized include, but are not limited to, fluorescein isothiocyanate; fluorescein dichlorotriazine and fluorinated analogs of fluorescein; naphthofluorescein carboxylic acid and its succinimidyl ester; carboxyrhodamine 6G; pyridyloxazole derivatives; Cy2, Cy3, Cy3.5, Cy5, Cy5.5, and Cy7; phycoerythrin; phycoerythrin-Cy conjugates; fluorescent species of succinimidyl esters, carboxylic acids, isothiocyanates, sulfonyl chlorides, and dansyl chlorides, including propionic acid succinimidyl esters, and pentanoic acid succinimidyl esters; succinimidyl esters of carboxytetramethylrhodamine; rhodamine Red-X succinimidyl ester; Texas Red sulfonyl chloride; Texas Red-X succinimidyl ester; Texas Red-X sodium tetrafluorophenol ester; Red-X; Texas Red dyes; tetramethylrhodamine; lissamine rhodamine B; tetramethylrhodamine; tetramethylrhodamine isothiocyanate; naphthofluoresceins); coumarin derivatives (e.g., hydroxycoumarin, aminocoumarin, and methoxycoumarin); pyrenes; acridines, pyridyloxazole derivatives; dapoxyl dyes; Cascade Blue and Yellow dyes; benzofuran isothiocyanates; sodium tetrafluorophenols; 4,4-difluoro-4-bora-3a,4a-diaza-s-indacene; Alexa fluors (e.g., 350, 430, 488, 532, 546, 555, 568, 594, 633, 647, 660, 680, 700, and 750); green fluorescent protein; and yellow fluorescent protein. The peak excitation and emission wavelengths will vary for these compounds and selection of a particular fluorescent probe for a particular application can be made in part based on excitation and/or emission wavelengths. In some embodiments, the fluorescent tag is fluorescein or one of its derivatives, rhodamine or one of its derivatives (including Tamra fluors, texas red and Rox), bodipy or a derivative thereof, an acridine, a coumarin, a pyrene, a benzanthracene or a cyanine (e.g., Cy3 and Cy5). In some embodiments, the fluorescent tag is a Tamra fluor. In some embodiments, the fluorescent tag is attached (e.g., to an oligonucleotide or a carrier macromolecule) by spacer arms of various lengths to reduce potential steric hindrance.

The term “therapeutically effective amount” as used herein refers to an amount which results in an improvement or remediation of the symptoms of the disease or condition. More particularly, the term “therapeutically effective amount” as used herein can refer to the amount of a pharmacological or therapeutic agent that will elicit a biological or medical response of a tissue, system, animal or mammal that is being sought by the administrator (such as a researcher, doctor or veterinarian) that includes alleviation of the symptoms of the condition or disease being treated and the prevention, slowing or halting of progression of one or more conditions.

### II. General Considerations

As described further hereinbelow in exemplary embodiments, the presently disclosed subject matter provides peptide-oligonucleotide conjugates and peptide-protein-conjugates that target receptors that mediate endocytosis, such as the αvβ3 integrin. The oligonucleotide is designed to correct an aberrant intron inserted into the luciferase gene of target cells that express the αvβ3 integrin. Successful delivery of the oligonucleotide to the nucleus is reflected by up-regulation of luciferase expression. The presently disclosed conjugates produce maximum effects that range between 30-60% of that seen by administration of complexes of oligonucleotides with cationic lipids (e.g., LIPOFECTAMINE™ 2000). However, the toxicities associated with cationic lipids has caused concern regarding their use for in vivo oligonucleotide delivery (see Lv et al. (2006) J. Control Release, 114, 100-109), while presently disclosed conjugates are relatively non-toxic, as shown herein, and can thus have potential for in vivo applications.

Further, previous reports have indicated that increased accumulation of anionic oligonucleotides caused by conjugation or complexation with polycationic peptides or polymers does not necessarily result in biologically effective delivery. See Juliano (2005) Curr. Opin. Mol. Ther., 7, 132-136; Abes et al. (2007) Biochem. Soc. Trans., 35, 53-55; Turner et al. (2005) Nucleic Acids Res., 33, 27-42; and Lundberg et al. (2007), Faseb J., 21, 2664-2671. In the cases of cationic lipids, or polymers with secondary amines such as PEI, their efficacy as nucleic acid carriers seems to be due to endosome-disrupting effects thus allowing release of the active material into the cytosol (see Hoekstra et al. (2007) Biochemical Society Transactions, 35, 68-71); however, such endosome damaging effects are unlikely when using RGD-containing peptides as delivery agents. There are several distinct mechanisms of endocytosis, each leading to endosomal trafficking patterns that have unique as well as overlapping features. See Perret et al. (2005) Current Opinion in Cell Biology, 17, 423-434. Integrins are known to recycle via internalization into endosomal compartments (see Caswell and Norman (2006) Traffic, 7, 14-21), but the exact pathways and mechanisms involved are not fully resolved. Several studies suggest that the αvβ3 integrin normally is internalized via caveolae and then takes the so-called ‘long loop’ Rab 11-dependent recycling pathway through the perinuclear recycling compartment. See Caswell and Norman (2006) Traffic, 7, 14-21; and White et al. (2007) J. Cell Biol., 177, 515-525. Without being bound to any one theory, it is possible that delivery to this compartment can afford increased opportunities for an RGD-oligonucleotide conjugate to escape from the interior of the endosomes.

II.A. Compositions

The presently disclosed subject matter relates to peptide and protein oligonucleotide conjugates for the delivery of the oligonucleotides via receptor mediated endocytosis. Further, the presently disclosed subject matter relates to the use of peptide-containing ligand groups that target specific receptors which mediate endocytosis.

Accordingly, in some embodiments, the presently disclosed subject matter provides a composition for delivering an oligonucleotide to a target cell through endocytosis, the composition comprising one or more ligand groups capable of mediating receptor endocytosis and one or more oligonucleotide groups that each comprise an oligonucleotide. The presently disclosed compositions are not limited to the use of oligonucleotides comprising neutral backbones (e.g., PNAs), but can be used to deliver any oligonucleotide, particularly naturally occurring oligonucleotides or other oligonucleotide derivatives comprising negatively charged backbones. In some embodiments, the oligonucleotide is capable of therapeutic activity. For example, the oligonucleotide can be selected from the group that includes, but is not limited to, an antisense RNA, a small interfering RNA (sRNA), and a micro RNA (miRNA) that selectively binds to an RNA in the target cell.

In some embodiments, it can be desirable to be able to track the delivery of the oligonucleotide to the cytoplasm or nucleus of the target cell. Thus, the oligonucleotide group can comprise a detectable tag (e.g., a fluorophore, luminophore, or radioisotope). In some embodiments, the detectable tag is a fluorophore, such as a Tamra fluor. The detectable tag can be attached (e.g., covalently directly or covalenty through a linker) to the oligonucleotide. The tag can be attached via any convenient method to the oligonucleotide backbone, to a base or sugar of one of the nucleic acid monomers, or to one end of the oligonucleotide. In some embodiments, the detectable tag is attached to a 3′ end of the oligonucleotide.

When the oligonucleotide is a therapeutic agent, the composition can be prepared to deliver a therapeutically effective amount of the oligonucleotide. The amount of oligonucleotide delivered can vary based on a number of factors, including, but not limited to, the number of oligonucleotide groups in the composition, the route of administration of the composition, and the dose or amount of composition used. In some embodiments, the composition is prepared for administration to a vertebrate subject. The subject can be a human or other mammal. Thus, the composition can be formulated for use in medical or veterinary settings. In some embodiments, the composition is prepared as a pharmaceutical formulation for administration to a human subject. Thus, the composition can be pharmaceutically acceptable for use in humans. The pharmaceutical formulation can be for intravenous, topical or parenteral administration. In some embodiments, the composition can be used in in vitro techniques, and the target cell is present in a cell culture. In such cases, the composition can comprise an oligonucleotide-ligand conjugate or oligonucleotide-carrier macromolecule-ligand conjugate present in a carrier liquid, such as water.

In some embodiments, the ligand groups comprise one or more peptide ligand that interacts with a target cell receptor that mediates endocytosis. The ligand can include more than one peptide ligand or can include a combination of different types of ligands, including non-peptide and/or small molecule ligands. The ligand groups can also include linker moieties. A number of ligands capable of mediating receptor endocytosis are known in the art. For example, useful peptide ligands include but are not limited to ligands for growth factor receptors such as EGF for the EGF receptor family member, EGFR1. Useful peptide ligands include but are not limited to peptide ligands for the chemokine receptor subfamily of GPCRs. CXCL12 can be used as a ligand for the receptor CXCR4, and CCL3 as a ligand for CCR5. Additional useful ligands include, but are not limited to, small organic molecule ligands for the chemokine subfamily of receptors. See, e.g., FD Goebel and B Juelq (2005) Infection, 5 408-10. Useful ligands also include small organic molecule nucleoside derivative ligands for the P2Y subfamily of GPCRs. See, e.g., Ko et al. (2007) J Med Chem, 50, 2030-2039. Other small molecule ligands include but are not limited to ligands of alpha and beta adrenergic receptors (other GPCRs). Many such ligands are currently in clinical use, for example, terbutaline as a beta agonist and phenylephrine as an alpha agonist. Still further useful ligands include peptide, peptidomimetic and non-peptide ligands for integrins such as a5b1, a4b1 and LFA-1. See, e.g., Simmons (2005) Curr. Opin. Pharmacology, 5, 398-404. Accordingly, in some embodiments, the different types of ligands include but are not limited to EGF, CXCL12, CCL3, small organic molecule ligands for chemokine receptors, small organic molecule ligands for the P2Y subfamily of GPCR receptors, small organic molecule ligands for alpha and beta adrenergic receptors, terbutaline, phenylephrine, and peptide, peptidomimetic and non-peptide ligands for integrins including a5b1, a4b1 and LFA-1. In some embodiments, the peptide ligand is a RGD peptide, such as, but not limited to, a cyclic RGD peptide. The RGD moiety serves to selectively bind the conjugate to the αvβ3 integrin receptor that is expressed on angiogenic endothelium cells and on some tumor cells.

In some embodiments, the one or more ligand groups and the one or more oligonucleotide groups are each attached to a carrier macromolecule. The carrier macromolecule can serve as a non-toxic platform for attaching any desired number of oligonucleotide groups and ligand groups. In particular, the attachment of several oligonucleotide groups can increase the therapeutic effictiveness of a single conjugate by delivering multiple copies of a therapuetic oligonucleotide to a single target cell. In some embodiments, the carrier macromolecule can be conjugated to between 2 and 10 oligonucleotide groups and between 2 and 10 ligand groups (e.g., 2, 3, 4, 5, 6, 7, 8, 9, or 10 groups). In some embodiments, the carrier macromolecule can be conjugated to more than 10 oligonucleotide or more than 10 ligand groups. In some embodiments, the carrier macromolecule is a protein. In some embodiments, the carrier macromolecule is a serum albumin protein. The protein can be chosen based on the intended target cell. For example, when the target cell is human, the carrier macromolecule can be human serum albumin.

The ligand and oligonucleotide groups can be attached to the carrier macromolecule via any convenient linker group. When the carrier macromolecule is a protein, thiol, hydroxyl or amino groups of amino acid side chains in the carrier macromolecule can be used as sites to attach the ligand or oligonucleotide groups. In some embodiments, thiol, hydroxyl, or amino groups of the protein can be used for the attachment of linker groups, which can be further reacted to moieties in the oligonucleotide or ligand groups.

In some embodiments, one or more ligand group comprises a PEG moiety, wherein the PEG moiety is attached to a carrier macromolecule (e.g., a protein). In some embodiments, the PEG moiety is attached to a protein through an amide linkage. In some embodiments, one or more ligand group comprises a cyclic RGD peptide attached to the PEG group through a maleimide or other suitable chemical group. The PEG moiety can be used to prevent or reduce non-specific interactions.

In some embodiments, the one or more oligonucleotide groups are attached to the carrier macromolecule through an alkylene linker group. In some embodiments, the alkylene linker group is —S—(CH₂)₆—. The sulfur atom of the —S—(CH₂)₆— group can form a bioreversible —S—S— bond with a thiol on the surface of the protein. In some embodiments, the ligand or oligonucleotide group can include an N-hydroxysuccinimide (NHS) moiety that can react with free amines on the surface of the protein to form an amide linkage. The linkages between the carrier macromolecule and the ligand group and/or the oligonucleotide group can be either bioreversible (i.e., can be cleaved under biologically relevant conditions, such as in vivo or in the cell cytoplasm) or be stable to cleavage under biological conditions.

In some embodiments, a ligand group is conjugated to an oligonucleotide group without an intervening carrier macromolecule. For example, the ligand group can comprise a maleimide moiety that can react with a thiol-terminated oligonucleotide to form a stable covalent linkage. Oligonucleotide-ligand groups can also be formed using other linkage chemistry. For example, the ligand can comprise an ester or NHS group that can be reacted with an amino-terminated oligonucleotide to form an amide. The linkage between the oligonucleotide group and the ligand group can be bioreversible or can be stable to cleavage under biological conditions.

In some embodiments, the peptide ligand is a multivalent peptide ligand including but not limited to a bi-, tri-, tetra-, penta-, hexa-, or an octa-valent peptide ligand. In some embodiments, the multivalent peptide ligand is a bicyclic RGD peptide. The bicyclic RGD peptide can be linked to a maleimide group. The maleimide group can in turn be linked to an oligonucleotide group through an alkylene linker group. The alkylene linker group can include a heteroatom. In some embodiments, the alkylene linker group is —S—(CH₂)₆—.

II.B. Methods of Delivering Oligonucleotides to Cells

The presently disclosed subject matter also provides methods of delivering an oligonucleotide to a cell through receptor mediated endocytosis. In some embodiments, the presently disclosed subject matter provides a method of delivering an oligonucleotide to a target cell through endocytosis, wherein the method comprises contacting the cell with a composition comprising one or more ligand groups capable of mediating receptor endocytosis and one or more oligonucleotide groups, wherein the one or more oligonucleotide groups each comprise an oligonucleotide, and wherein the target cell comprises one or more receptors capable of mediating receptor endocytosis in response to the one or more ligand groups, thereby actively transporting the oligonucleotide into the target cell. The composition can comprise a ligand group conjugated to an oligonucleotide group or a carrier molecule conjugated to both one or more ligand groups and one or more oligonucleotide groups. Thus, in some embodiments, the methods of the presently disclosed subject matter can be used to deliver an antisense RNA, a siRNA, or a miRNA to the target cell. The delivery of the oligonucleotide can be used to regulate gene expression in the cell, either as part of a therapeutic method or as part of a research or diagnostic technique.

In some embodiments, the target cell is present in a subject, and contacting the target cell with the composition comprises administering a therapeutically effective amount of the composition to the subject. The composition can be administered via any suitable route, including, but not limited to intravenous, interperitoneal, parenteral, topical, intranasal, inhalation, or buccal routes.

### III. Formulations

A therapeutic composition as described herein comprises in some embodiments a composition that includes a pharmaceutically acceptable carrier. Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compositions used in the methods can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

Suitable methods for administering to a composition or formulation to a subject include but are not limited to systemic administration, parenteral administration (including intravascular, intramuscular, intraarterial administration), buccal delivery, subcutaneous administration, inhalation, intratracheal installation, surgical implantation, transdermal delivery, local injection, and hyper-velocity injection/bombardment. Where applicable, continuous infusion can enhance conjugate delivery to a target site.

The amount and timing of conjugate administered can, of course, be dependent on the subject being treated, the route of administration, on the pharmacokinetic properties of the conjugate, and on the judgment of the prescribing physician. In considering the degree of treatment desired, the physician can balance a variety of factors such as age and weight of the subject, presence of preexisting disease, as well as presence of other diseases.

The therapeutically effective dosage of any conjugate, the use of which is within the scope of embodiments described herein, can vary somewhat from compound to compound, and subject to subject, and can depend upon the condition of the subject and the route of delivery.

The pharmaceutical formulations can comprise a conjugate described herein or a pharmaceutically acceptable salt thereof, in any pharmaceutically acceptable carrier. If a solution is desired, water is the carrier of choice with respect to water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and typically by filtration through a 0.22-micron filter. Subsequent to sterilization, the solution can be dispensed into appropriate receptacles, such as depyrogenated glass vials. The dispensing is optionally done by an aseptic method. Sterilized closures can then be placed on the vials and, if desired, the vial contents can be lyophilized.

In addition to the conjugates or their salts, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. An antimicrobial preservative is typically employed when the formulation is placed in a vial designed for multi-dose use. The pharmaceutical formulations described herein can be lyophilized using techniques well known in the art.

In some embodiments of the subject matter described herein, there is provided an injectable, stable, sterile formulation comprising a conjugate as described herein, or a salt thereof, in a unit dosage form in a sealed container. The conjugate or salt is provided in the form of a lyophilizate, which is capable of being reconstituted with a suitable pharmaceutically acceptable carrier to form a liquid formulation suitable for injection thereof into a subject. When the conjugate or salt is substantially water-insoluble, a sufficient amount of emulsifying agent, which is physiologically acceptable, can be employed in sufficient quantity to emulsify the conjugate or salt in an aqueous carrier. Particularly useful emulsifying agents include phosphatidyl cholines and lecithin.

Pharmaceutical formulations also are provided which are suitable for administration as an aerosol by inhalation. These formulations comprise a solution or suspension of a desired conjugate described herein or a salt thereof, or a plurality of solid particles of the conjugate or salt. The desired formulation can be placed in a small chamber and nebulized. Nebulization can be accomplished by compressed air or by ultrasonic energy to form a plurality of liquid droplets or solid particles comprising the conjugates or salts. The liquid droplets or solid particles should have a particle size in the range of about 0.5 to about 10 microns, and optionally from about 0.5 to about 5 microns. The solid particles can be obtained by processing the solid compound or a salt thereof, in any appropriate manner known in the art, such as by micronization. Optionally, the size of the solid particles or droplets can be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

When the pharmaceutical formulation suitable for administration as an aerosol is in the form of a liquid, the formulation can comprise a water-soluble conjugate in a carrier that comprises water. A surfactant can be present, which lowers the surface tension of the formulation sufficiently to result in the formation of droplets within the desired size range when subjected to nebulization.

As indicated, both water-soluble and water-insoluble conjugates are provided. As used herein, the term “water-soluble” is meant to define any composition that is soluble in water in an amount of about 50 mg/mL, or greater. Also, as used herein, the term “water-insoluble” is meant to define any composition that has a solubility in water of less than about 20 mg/mL. In some embodiments, water-soluble conjugates or salts can be desirable whereas in other embodiments water-insoluble conjugates or salts likewise can be desirable.

The term “pharmaceutically acceptable salts” as used herein refers to those salts which are, within the scope of sound medical judgment, suitable for use in contact with subjects (e.g., human subjects) without undue toxicity, irritation, allergic response, and the like, commensurate with a reasonable benefit/risk ratio, and effective for their intended use, as well as the zwitterionic forms, where possible, of the conjugates of the presently disclosed subject matter.

Thus, the term “salts” refers to the relatively non-toxic, inorganic and organic acid addition salts of conjugates of the presently disclosed subject matter. These salts can be prepared in situ during the final isolation and purification of the conjugates or by separately reacting the purified conjugate in its free base form with a suitable organic or inorganic acid and isolating the salt thus formed.

The base addition salts of acidic conjugates are prepared by contacting the free acid form with a sufficient amount of the desired base to produce the salt in the conventional manner. The free acid form can be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the presently disclosed subject matter.

Salts can be prepared from inorganic acids sulfate, pyrosulfate, bisulfate, sulfite, bisuffite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

With respect to the methods of the presently disclosed subject matter, a preferred subject is a vertebrate subject. A preferred vertebrate is warm-blooded; a preferred warm-blooded vertebrate is a mammal. The subject treated by the presently disclosed methods is desirably a human, although it is to be understood that the principles of the presently disclosed subject matter indicate effectiveness with respect to all vertebrate species which are included in the term “subject.” In this context, a vertebrate is understood to be any vertebrate species in which treatment of a disorder is desirable. As used herein “subject” includes both human and animal subjects. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos or as pets, as well as fowl, and more particularly domesticated fowl, i.e., poultry, such as turkeys, chickens, ducks, geese, guinea fowl, and the like, as they are also of economical importance to humans. Thus, also provided is the treatment of livestock, including, but not limited to, domesticated swine, ruminants, ungulates, horses (including race horses), poultry, and the like.

### EXAMPLES

The presently disclosed subject matter will now be described more fully hereinafter with reference to the accompanying Examples in which representative embodiments are shown. The presently disclosed subject matter can, however, be embodied in different forms and should not be construed as limited to the embodiments set forth herein.

Disclosed herein in some embodiments are the preparation and characterization of conjugates between an anionic antisense oligonucleotide and a bivalent bicyclic RGD peptide that binds with high affinity to the αvβ3 integrin. See Chen et al. (2005) J. Med. Chem., 48, 1098-1106. Members of the integrin family of cell surface receptors provide structural linkages between the extracellular matrix and the cytoskeleton, but are also importantly involved in the control of signal transduction pathways. See Juliano (2002) Annu Rev. Phamacol. Toxicol., 42, 283-323. The αvβ3 integrin is of particular interest in cancer since it is highly expressed both in angiogenic endothelial cells and certain types of malignant cells. See Stupack and Cheresh (2004) Curr. Top. Dev. Biol., 64, 207-323. Thus it can provide an approach to selectively target growth-regulatory oligonucleotides to tumors or tumor vasculature. The bivalent peptide was coupled to a “splice shifting oligonucleotide” (SSO) designed to correct splicing of an aberrant intron inserted into the firefly luciferase reporter gene. See Kanq et al. (1998) Biochemistry, 37, 6235-6239. Thus successful delivery of the SSO to the cell nucleus results in up-regulation of luciferase activity. Using this approach, it was shown that the bivalent RGD peptide can effectively deliver the SSO to αvβ3-expressing melanoma cells in culture via a receptor mediated uptake process.

### Example 1

### General Methods for Peptide-Oligonucleotide Conjugates

The sequence termed oligonucleotide 623 (5′-GTT ATT CTT TAG AAT GGT GC-3′; SEQ ID NO: 1) and its conjugates, as well as mismatched control oligonucleotide, referred to as 5MM623 (5′-GTA ATT ATT TAT AAT CGT CC-3′; SEQ ID NO: 2) and its conjugates, were prepared as described below. All oligonucleotides include 2′-OMe ribose residues with phosphorothioate backbones.

Phosphoramidites, Controlled Pore Glass (CPG) Supports, and Other Reagents. 5′-O-(4,4′-Dimethoxytrityl)-N-phenoxyacetyl-2′-O-methyl-Adenosine-3′-O-((β-cyanoethyl)-(N,N-diisopropyl))-phosphoramidite [2′-OMe-Pac-A-CE Phosphoramidite], 5′-O-(4,4′-Dimethoxytrityl)-N-2-isopropylphenoxyacetyl-2′-O-methyl-Guanosine-3′-O-((β-cyanoethyl)-(N,N-diisopropyl))-phosphoramidite [2′-OMe-iPr-Pac-G-CE Phosphoramidite], 5′-O-(4,4′-Dimethoxytrityl)-N-acetyl-Cytidine-2′-O-methyl-3′-O—W3-cyanoethyl)-(N,N-diisopropyl))-phosphoramidite [2′-OMe-Ac-C-CE Phosphoramidite], 1-O-(4,4′-Dimethoxytrityl)-hexyldisulfide-1′-((β-cyanoethyl)-(N,N-diisopropyl))-phosphoramidite [Thiol-Modifier C6 S—S], 1-O— (4,4′-Dimethoxytrityl)-3-(O—(N-carboxy-(Tetramethyl-rhodamine)-3-aminopropyl))-propyl-2-O-succinoyl-long chain alkylamino-CPG [3′-Tamra CPG], 3H-1,2-Benzodithiole-3-one-1,1-dioxide [Beaucage Reagent], and other reagents for DNA synthesis were purchased from Glen Research (Sterling, Va.). 5′-O-(4,4′-Dimethoxytrityl)-5-methyluridine-2′-O-methyl-3′-O-((β-cyanoethyl)-(N,N-diisopropyl))-phosphoramidite [2′-OMe-T-CE Phosphoramidite] was purchased from Chemgenes (Ashland, Mass., United States of America).

Synthesis, Cleavage and De-protection of Oligonucleotides. Oligonucleotides were synthesized using phosphoramidites of the ultraMILD protected bases indicated above on a 1 pmole scale on 3′-Tamra CPG supports (500A) using a AB 3400 DNA synthesizer (Applied Biosystems, Foster City, Calif., United States of America). The coupling times for the phosphoramidites of ultraMILD protecting bases and 5′-thiol linker were 360 and 600 s, respectively. 5-Ethylthio-1H-tetrazole was used as an activator (0.25 M solution in acetonitrile), 5% phenoxyacetic anhydride in tetrahydrofuran/pyridine as a CAP mix A, and Beaucage reagent was used to introduce the internucleotide phosphorothioate backbone during oligonucleotide synthesis. A 5′-thiol linker was introduced at the 5′-end of the oligonucleotide.

Oligonucleotides were simultaneously cleaved from the CPG support and deprotected using a mixture of tert-butylamine:methanol:water (1:1:2) at 55° C. for 8 hours. Prior to deprotection, the CPG supports were treated with a 10% solution of diethylamine in acetonitrile. This removes cyanoethyl protection and prevents elimination of the 3′-Tamra linker from the oligonucleotide. This was done with disposable syringes using 2×1 mL solution for five minutes each followed by washing with acetonitrile and drying the support with a stream of argon gas. The CPG support was then transferred to a vial and 2 mL deprotection solution was added and heated for 8 h at 55° C. The oligonucleotide solution was immediately evaporated to dryness, and resuspended in 0.1 M TEAA buffer for purification.

Purification and Structure Determination. Purification of the oligonucleotides was carried out by reverse-phase HPLC using a ZORBAX™ 300 SB-C18 column (9.4 mm×25 cm; Agilent Technologies, Santa Clara, Calif., United States of America) on a Varian ProStar/Dynamax HPLC system (Varian Inc., Palo Alto, Calif., United States of America) with a ProStar 335 PDA detector (Varian Inc., Palo Alto, Calif., United States of America). HPLC conditions were as follows: linear gradient, % buffer B=10-30%/20 min, ˜100%130 min, 4 mL/min; buffer A contained 0.1 M TEAA, pH 7.0 and buffer B: acetonitrile; UV monitor: 254 and 550 nm (λₘₐₓ for Tamra). The oligos were collected and lyophilized. Structures of the oligonucleotides were determined using matrix-assisted laser desorption ionization time-of-flight (MALDI-TOF) mass spectroscopy in a positive ion mode on a Voyager™ Applied Biosystem instrument (Applied Biosystems, Foster City, Calif., United States of America). The matrix used for preparing the oligonucleotide samples was a mixture of 3-hydroxypicolinic acid (50 mg/mL in 50% aqueous acetonitrile) and diammonium hydrogen citrate (50 mg/mL in HPLC grade water. The accuracy of the mass measurement was ±0.02%.

Preparation of 5′-thiol Oligonucleotides. The 5′-thiol functionality was generated by treating the disulfide bond of the oligonucleotide with 100 mM of aqueous DTT in 0.1M TEAA buffer containing 1% triethylamine. After overnight incubation, the reaction mixture was desalted through a Sep-PAK® C18 cartridge (Waters Corporation, Milford, Mass., United States of America), and any residual amount of DTT was removed by washing with 5% acetonitrile in a 0.1 M TEAA buffer. Finally, the Tamra-623-SH oligonucleotide was eluted with 50% aqueous acetonitrile and directly used for the conjugation reaction. The structure of the thiol-containing oligonucleotide was confirmed by MALDI-TOF as described above.

Synthesis of Bivalent RGD Peptide-oligonucleotide Conjugates. The synthesis and characterization of similar bivalent RGD peptides has been described elsewhere (see Chen et al., (2005) J. Med. Chem., 48, 1098-1106); however in the present case a maleimide linker was incorporated so as to allow conjugation with the oligonucleotide. The cyclic RGD dimer (10 μmol) was reacted with maleimide N-hydroxysuccinimide (NHS) ester (15 μmol) in borate buffer (0.05 N, pH 8.5) at room temperature. After 2 h, RGD-maleimide was isolated by semi-preparative HPLC with a 70% yield. Mass spectrometry analysis (MALDI-TOFMS: 1515.72 for [MH] (C₆₇H₉₅N₂₀O₂₁, calculated [MW] 1515.69)) confirmed the product identification. Thiol oligonucleotides (316 nmoles in 50% aqueous CH₃CN) were reacted with the maleimide-containing bivalent RGD peptide (475 nmoles in water) in a reaction buffer (final salt concentration adjusted to 400 mM KCl, 40% aqueous CH₃CN). The reaction mixture was vortexed and allowed to stand for 3 h, and purified by HPLC using a 1 mL Resource Q column (GE Healthcare, Chalfont St. Giles, United Kingdom) following a published method. See Turner et al. (2005) Nucleic Acids Res., 33, 27-42. Buffers were as follows: buffer A, 20 mM Tris-HCl (pH 6.8), 50% formamide; buffer B, 20 mM Tris-HCl (pH 6.8), 400 mM NaClO4, 50% formamide; linear gradient, % buffer B=0˜100%/20 min, 3 mL/min; UV monitor, 254 nm and 550 nm. The purified conjugates were dialyzed versus milli-Q water, and analyzed by MALDI-TOF using a matrix which was a mixture of 2,6-dihydroxyacetophenon (20 mg/mL) and diammonium hydrogen citrate (40 mg/mL) in 50% aqueous methanol. Various versions (see Example 2, below) of the bivalent RGD peptide-623 conjugate were made including conjugates with or without the Tamra fluorophore, as well as control conjugates having an oligonucleotide with multiple (5) mismatches (i.e., 5MM623, SEQ ID NO: 2).

Cell Lines and Plasmids: A375SM melanoma cells were obtained from Dr. J. Bear (University of North Carolina, Chapel Hill, N.C., United States of America), and were cultured in Dulbecco's minimum essential medium (DMEM; Invitrogen, Carlsbad, Calif., United States of America) supplemented with L-glutamine and 10% fetal bovine serum (FBS). Plasmid pLuc/705, containing an aberrant intron inserted into the firefly luciferase coding sequence, was obtained from Dr. R. Kole (University of North Carolina, Chapel Hill, N.C., United States of America). See Kang et al. (1998) Biochemistry, 37, 6235-6239. Stable transfectants were obtained by cotransfecting A375SM cells with one part of hygromycin resistant plasmid pcDNA3.1(+)/hygro (Invitrogen, Carlsbad, Calif., United States of America) and ten parts of pLuc/705 using LIPOFECTAMINE™ 2000 (Invitrogen, Carlsbad, Calif., United States of America) as per manufacturer's instructions. Selection was carried out in DMEM containing 200 μg/mL hygromycin and 10% FBS. The resulting pool of hygromycin resistant cells was referred to as A375SM-Luc705-B.

Oligonucleotide treatment and luciferase assay: A375SM-Luc705-B cells were plated on 12 well plates (at 1.0 or 1.5×10⁵ cells per well in various experiments) in DMEM supplemented with 10% FBS. The following day, medium was changed to reduced serum OPTI-MEM I (Invitrogen, Carlsbad, Calif., United States of America). Cells were treated with either free 623 oligonucleotide, 623 complexed with LIPOFECTAMINE™ 2000 (Invitrogen, Carlsbad, Calif., United States of America) as per manufacturer's instruction, or RGD-623 conjugate, or with mismatched control oligonucleotides. Four hours after treatment, 1% FBS was added to each well. Twenty four hours after oligonucleotide treatment, medium was replaced with DMEM containing 1% FBS, and at various times thereafter cell lysates were collected for luciferase assay.

Cells were usually harvested 48 hours after oligonucleotide treatment, or at times indicated in the figures, and activity determined using a Luciferase assay kit (Promega, Madison, Wis., United States of America). Measurements were performed on a Monolight 2010 instrument (Analytical Luminescence Laboratory, San Diego, Calif., United States of America). In some cases the effects of the RGD-623 conjugate were evaluated in the presence of free monovalent cyclic RGDfV peptide (Anaspec, San Jose, Calif., United States of America).

Cell Uptake, Confocal Fluorescence Microscopy, and Flow Cytometry: Total cellular uptake of Tamra-labeled oligonucleotides was monitored using a Nanodrop microfluorimeter (Nanodrop Technologies, Wilmington, Del., United States of America). After treatment with oligonucleotides the cells were lysed in a mild non-ionic detergent buffer and the Tamra fluorescence (emission 583 nm) was quantitated based on a linear standard curve of unconjugated Tamra in buffer. Intracellular distribution of Tamra-labeled oligonucleotides was examined using an Olympus Confocal FV300 fluorescent microscope (Olympus America Inc., Center Valley, Pa., United States of America) with 60X-oil immersion objective, as previously described. See Astriab-Fisher et al. (2004) Biochem. Pharmacol., 68, 403-407. Expression levels of αvβ3 were monitored by immunostaining with an anti-human-αvβ3 monoclonal (Chemicon, Temecula, Calif., United States of America) followed by an Alexa 488 rabbit antimouse secondary antibody, with analysis by flow cytometry using a DakoCytomation (Glostrop, Denmark) machine.

Toxicity Studies: Cells were treated with various concentrations of oligonucleotides or conjugates under the same conditions as used for the luciferase induction experiments. After 48 h in medium plus 1% FBS, cells were trypsinized and viable cells were counted in an electronic particle counter.

### Example 2

### Synthesis and Characterization of a Bicyclic RGD Peptide-Oligonucleotide Conjugate

Oligonucleotide 623 is a 2′-O-Me phosphorothioate sequence that is designed to correct splicing of an aberrant intron from thalassemic hemoglobin; this intron can be inserted into various reporter genes, such as luciferase, and correction of the splicing defect results in up-regulation of gene expression (41,42). See Kole et al. (2004) Oligonucleotides, 14, 65-74; and Resina et al. (2007) J. Gene Med., 9, 498-510. This provides a sensitive and convenient positive readout for monitoring delivery of splice switching oligonucleotides (SSOs) to the cell nucleus, the compartment where splicing takes place. It also avoids the typical pitfalls of many assays of antisense or siRNA effects that rely on inhibition of gene expression and can thus be confounded by non-specific toxicities. The oligonucleotide is linked to a peptide that contains two modules of a cyclic RGD sequence. This allows the bivalent peptide to bind with high affinity to the αvβ3 integrin (see Chen et al. (2005) J. Med. Chem., 48, 1098-1106), a cell surface receptor that is highly expressed in angiogenic endothelial cells and certain types of tumor cells, including the A375 melanoma cells used here.

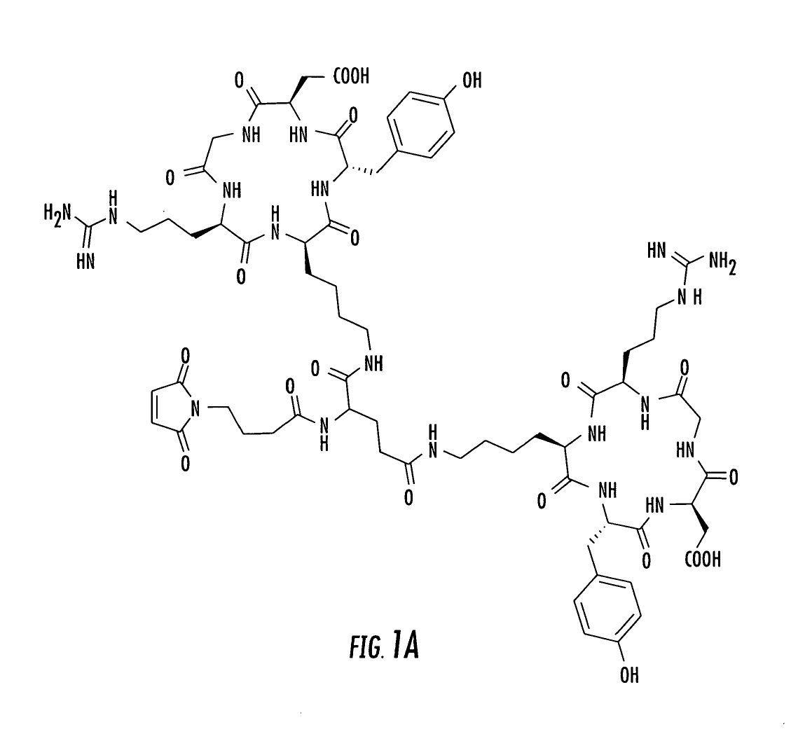

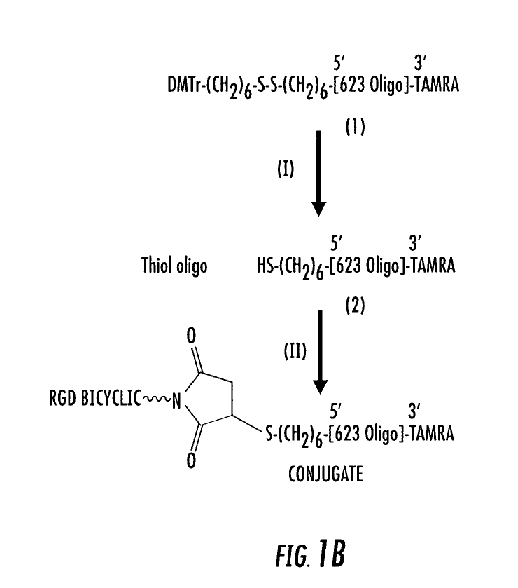

The chemical structure of the bicyclic RGD peptide used herein is shown in FIG. 1A. The peptide contains a maleimide functionality which can be coupled with 5′-thiol oligonucleotide 623 via the Michael addition reaction according to the steps outlined in FIG. 1B. A Tamra fluorophore was introduced at the 3′-end of the oligonucleotide and a thiol C6 S—S linker was introduced at the 5′-end. The DMTr-(CH₂)₆—S—S—(CH₂)₆-[623]-Tamra oligonucleotide (1) was purified by RP-HPLC, and its disulfide bridges were reduced with DTT solution to generate highly reactive 5′-HS—(CH₂)₆-[623]-Tamra oligonucleotide (2). Reagent conditions (I) were 100 mM DTT, 0.1M TEAA buffer, and 1% triethylamine. Reagent conditions (II) were maleimide-bicyclic-RGD peptide in H₂O (1.5 equivalents), 400 mM KCl, 40% CH₃CN, 3 h, RT.

The peptide conjugation reaction occurred between the maleimide of the bicyclic RGD peptide and the 5′ thiol (—SH) of the oligonucleotide (2). The reaction proceeded efficiently and more than 95% of the starting oligonucleotide (2) was converted into conjugates with bicyclic RGD. The conjugates were purified by ion-exchange chromatography (Resource™ Q column, GE Healthcare, Chalfont St. Giles, United Kingdom) under highly denaturing conditions to avoid any sort of precipitation, although this was not expected to be a problem for this type of peptide. HPLC profiles for the purified bicyclic RGD-623 conjugate and for the thiol oligonucleotide (2) are given in FIG. 1C, which shows that the conjugate peak is clearly resolved from the starting thiol oligonucleotide. After dialysis and lyophilization, the conjugate re-dissolved in sterile water without any difficulty. Similar approaches were used for the preparation of other oligonucleotides and conjugates. In each case the structure of the final product was confirmed by MALDI-TOF mass spectroscopy. The structures and characteristics of the various exemplary oligonucleotides and conjugates synthesized are given in Tables 1 and 2, below.

TABLE 1
Examples of Oligonucleotide Sequences

| Oligo | Sequences* |
|---|---|
| 623 | 5′-GTTATTCTTTAGAATGGT GC-3′ |
| (SEQ ID NO: 1) | |
| 623-Tamra | 5′-GTTATTCTTTAGAATGGTGC-Tamra-3′ |
| 623-SH | 5′-HS-(CH₂)₆-GTTATTCTTTAGAATGGTGC-3′ |
| Tamra-623-SH | 5′-HS-(CH₂)₆-GTTATTCTTTAGAATGGTGC- |
| | Tamra-3′ |
| 5MM623 or MM | 5′-GTAATTATTTATAATCGTCC-3′ |
| (SEQ ID NO: 2) | |
| 5MM623-Tamra | 5′-GTAATTATTTATAATCGTCC-Tamra-3′ |
| 5MM623-SH | 5′-HS-(CH₂)₆-GTAATTATTTATAATCGTCC-3′ |
| Tamra-5MM623-SH | 5′-HS-(CH₂)₆-GTAATTATTTATAATCGTCC- |
| | Tamra-3′ |

*All oligonucleotides consist of 2′-OMe ribose residues with phosphorothioate backbone.

TABLE 2
Oligonucleotides/Conjugates Molecular Weights.

| Oligonucleotide/Conjugate | MW_{calcd} | MW_{found} |
|---|---|---|
| 623-Tamra | 7691.97 | 7691.80 |
| HS-623-Tamra | 7910.74 | 7912.01 |
| BivalentRGD-Mal-S—(CH₂)₆-623-Tamra | 9427.32 | 9427.43 |
| 5MM623-Tamra | 7620.00 | 7620.20 |
| HS-5MM623-Tamra | 7833.26 | 7833.50 |
| Bivalent-RGD-Mal-S—(CH₂)₆-5MM623-Tamra | 9347.95 | 9345.50 |

### Example 3

### Dose-Response Studies

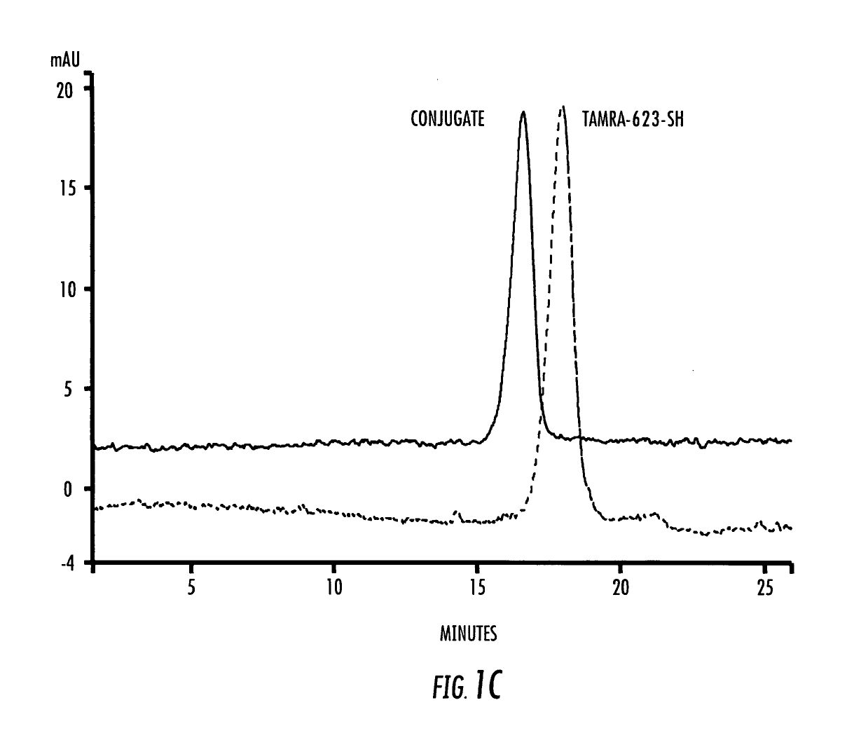

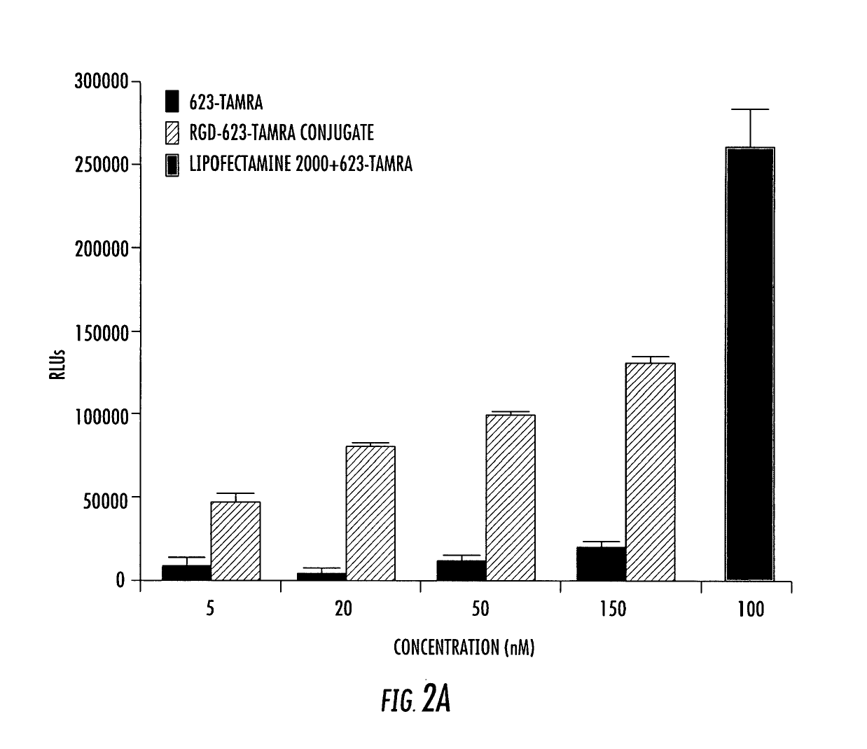

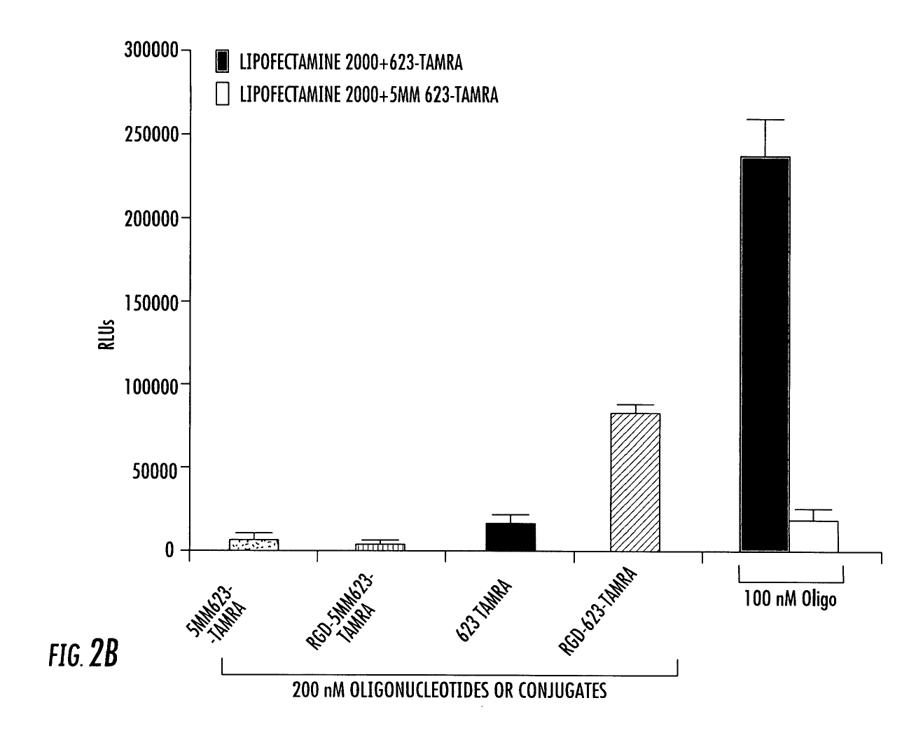

The RGD-623 conjugate, as well as 623-Tamra, or 623 complexed with LIPOFECTAMINE™ 2000 (Invitrogen, Carlsbad, Calif., United States of America) were incubated with A375SM-Luc705-B cells as described in Methods and the cells were tested for luciferase expression. As seen in FIG. 2A, the RGD-623 conjugate produced a significant increase in luciferase expression while the 623-Tamra did not. The effect of higher concentrations of RGD-623 was approximately 50% of that produced by the 623/cationic lipid complex. No more than 100 nM oligonucleotide with the LIPOFECTAMINE™ 2000 was used because the complex material became quite toxic at higher concentrations of oligonucleotide. Since substantial effects were observed at 50 nM of the RGD-623 conjugate, lower concentrations were studied as well. As seen in FIG. 2B, significant effects were observed even at 5 nM concentration. The data suggests that the effect of the RGD-623 conjugate rises rapidly between zero and 100 nM and then begins to plateau. This behavior is consistent with the known affinities of the bivalent RGD peptide and its conjugates for the αvβ3 integrin, these being approximately in the 15-30 nM range. See Chen et al. (2005) J Med. Chem., 48, 1098-1106. Use of a control oligonucleotide (5MM623-Tamra) or a control RGD conjugate (comprising 5MM623-Tamra), each having five sequence mismatches, failed to produce an increase in luciferase expression (FIG. 2C), indicating that the luciferase response depends on specific base-pairing.

Additional studies indicated that the presence of the Tamra fluorophore did not affect the action of 623 (SEQ ID NO: 1), delivered either as the RGD conjugate or via lipofection, at concentrations up to 75 nM. At concentrations above 100 nM, there was a slight augmentation of effect by Tamra. Without being bound to any one theory, this is believed to be due to some hydrophobic binding of the conjugate to the cell membrane.

### Example 4

### Time-Response Studies

The kinetics and duration of action of the RGD-623 conjugate were examined by harvesting the cells at various times after the period of exposure to the oligonucleotide. As seen in FIG. 3, there was a striking difference between the kinetics of the RGD-623 conjugate and the LIPOFECTAMINE™ 2000/623 complex. The effect of the RGD-623 conjugate on luciferase expression rose gradually with time and reached a maximum at 72 h (48 h after removal of the oligonucleotide). In contrast, the effect of the LIPOFECTAMINE™ 2000/623 complex was greatest at very early time points after exposure to the oligonucleotide and declined steadily thereafter. This indicates that the two modes of delivery operate by very different mechanisms. The oligonucleotide delivered via cationic lipids seems to go directly to the nucleus, while that delivered via the peptide-conjugate seems to traffic through other intracellular compartments and only gradually reach the nucleus.

### Example 5

### Total Cellular Uptake

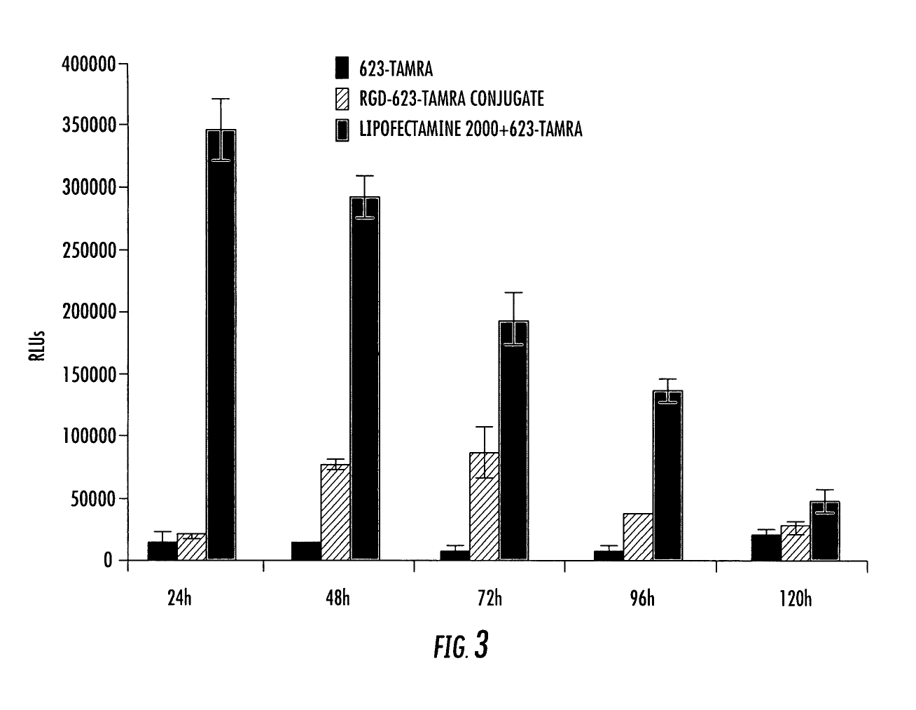

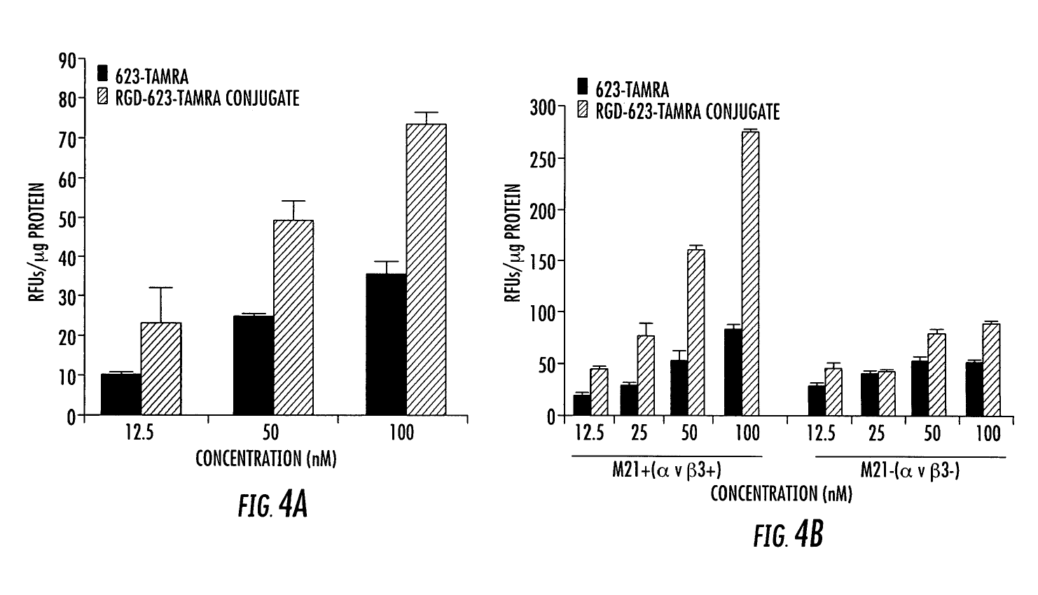

Total cellular uptake of 623-Tamra or its RGD-conjugate were evaluated by incubating cells with various concentrations of these molecules and then measuring total cell-associated fluorescence, as described in Example 1. As seen in FIG. 4A, there was approximately a 2-fold higher uptake of the RGD-conjugate as compared to the unconjugated oligonucleotide. In general, the increased uptake could be blocked by co-incubation with excess free cyclic RGD peptide suggesting the involvement of the αvβ3 integrin. As indicated in FIG. 4B, there was about 3 times more uptake of the RGD-conjugate by M21+ cells as compared to M21− cells. In the M21− cells there was slightly greater uptake of the RGD-623-Tamra conjugate as compared to the 623-Tamra control. This could be due to the presence of other RGD-binding integrins (e.g., a5β1 or αvβ1) that could associate with the conjugate, even at a lower affinity.

To determine whether incubation with RGD-623-Tamra down-regulates expression of its integrin receptor, cell surface levels of αvβ3 before and after 24 h exposure to RGD-conjugate. As shown in FIG. 4C, exposure of cells to 200 nM of RGD-623-Tamra or 623-Tamra had no effect on the level of surface αvβ3 expression. Thus, without being bound to any one theory, the data of FIGS. 4A-4C appears to indicate that a significant portion of the uptake of the RGD-oligonucleotide conjugate occurs via the αvβ3 from the cell surface.

### Example 6

### Inhibition with Excess RGD Peptide

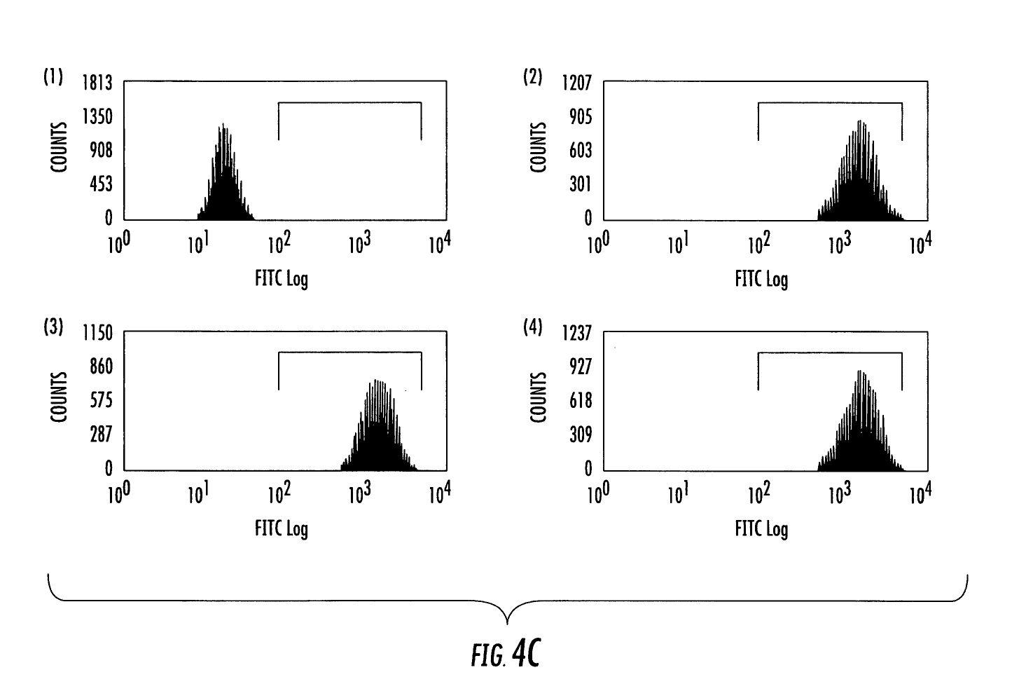

The conclusion that the RGD-623 conjugates enter the cell via receptor mediated endocytosis involving the αvβ3 integrin was tested. If that were the case, then the effects of RGD-623 should be blocked by co-incubation with excess amounts of another ligand that binds to the same site on αvβ3. As a blocking agent, a cyclic RGD peptide (RGDfV) that is known to be a selective inhibitor of αvβ3, was utilized. See Friedlander et al. (1996) Proc. Natl. Acad. Sci. USA, 93, 9764-9769; and Mitra et al. (2006) J. Control. Release, 114, 175-183. As shown in FIG. 5, co-incubation with increasing concentrations of this peptide led to a progressive inhibition of the effect of RGD-623-Tamra on luciferase expression. This observation supports the concept that the effects of RGD-623-Tamra on splicing depend on its initial uptake via the αvβ3 receptor.

### Example 7

### Subcellular Distribution

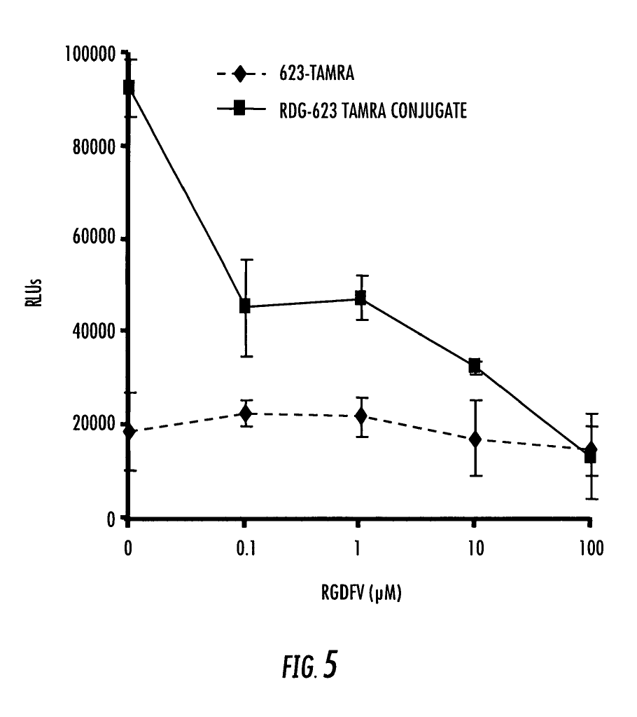

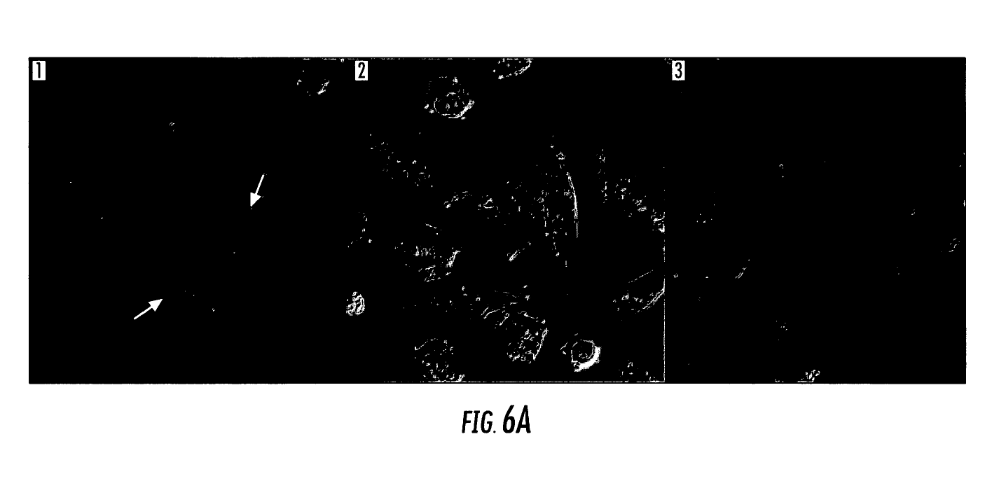

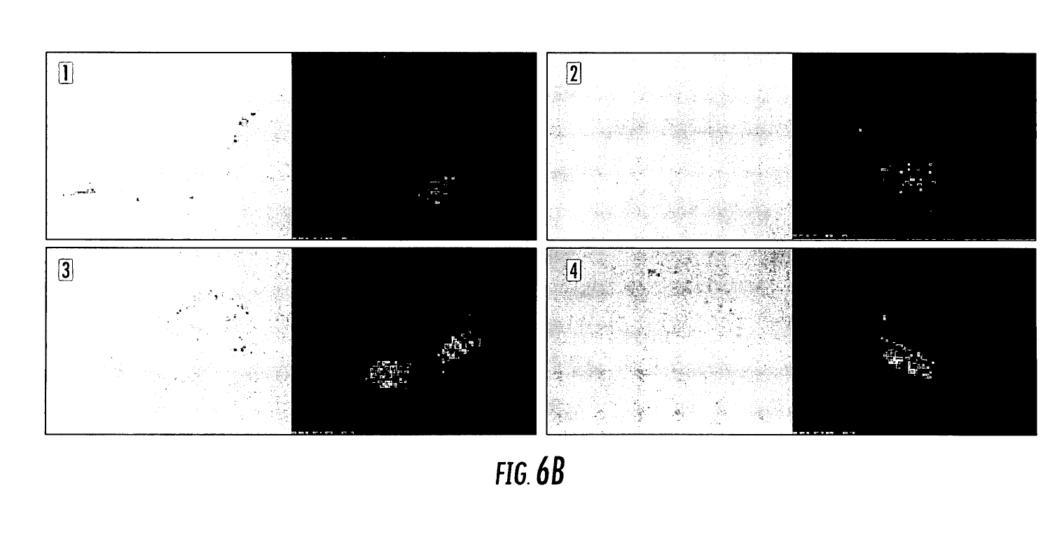

Subcellular distribution of the Tamra-labeled oligonucleotides in living cells was examined by confocal fluorescence microscopy. As shown in FIG. 6A, in cells treated with 623-Tamra complexed with LIPOFECTAMINE™ 2000 (Invitrogen, Carlsbad, Calif., United States of America), a substantial amount of cell uptake was seen. This was primarily associated with cytoplasmic vesicles, while a fraction of the cells clearly had fluorescence within the cell nucleus. See FIG. 6A, dark arrows. In the case of ‘free’ 623-Tamra, relatively little uptake was observed, consistent with the findings in Example 5. With the RGD-623-Tamra conjugate, substantial cellular uptake was observed; however, it was primarily associated with cytoplasmic vesicles and there was no readily observable nuclear fluorescence. Co-incubation of the RGD-623-Tamra conjugate with free RGD peptide substantially reduced total cell associated fluorescence in the confocal images (data not shown).

A preliminary investigation of the subcellular trafficking of the RGD-conjugate was undertaken. Using live cells, the co-localization of the RGD-623-Tamra conjugate with transferrin or dextran, known markers, respectively for clathrin-coated vesicle or smooth vesicle endocytosis, was studied. At 2 h, there was no co-localization of the RGD conjugate with transferrin, but there was significant co-localization with dextran. See FIG. 6B, panels 1 and 2. After 24 h, there was substantial co-localization of the RGD-conjugate with either the transferrin or dextran markers. The data suggests that the RGD-oligonucleotide conjugate initially enters cells via a non-clathrin-mediated endocytic process, but eventually trafficks through various endomembrane compartments including some that can also be used by transferrin.

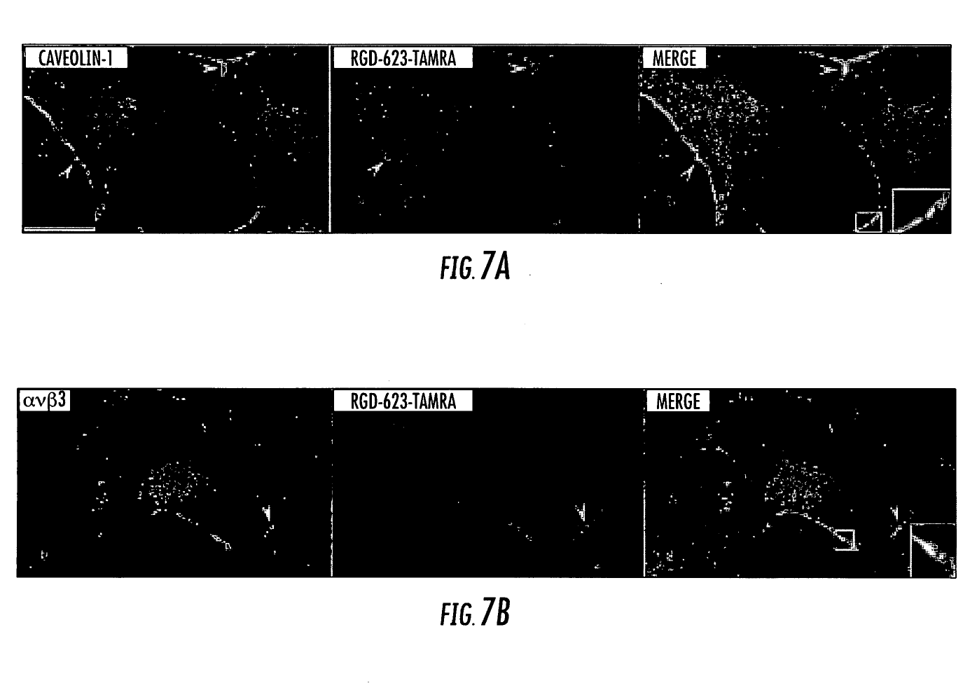

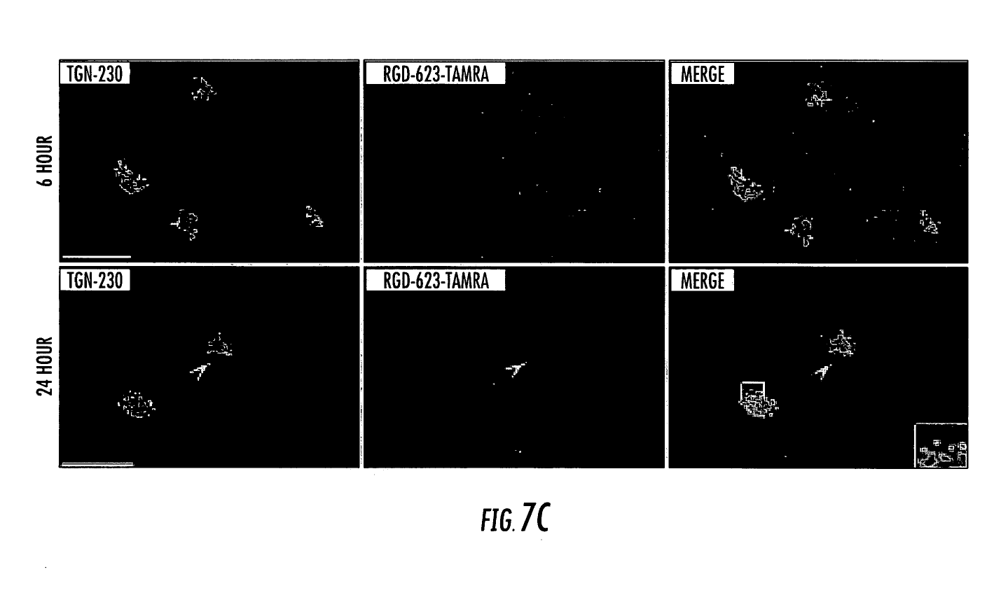

Pursing the subcellular fate of the RGD-conjugates further, its distribution was compared to well-known markers for several endomembrane compartments, using immuno-localization in fixed and permeabilized cells. The conjugate was not found to re-localize upon fixation and permeabilization of cells. As shown in FIG. 7A, at times between 2 and 6 h, there was significant co-localization of RGD-623-Tamra with caveolin-1. Long strands of caveolin-1-positive endosomes were observed near cell edges, and some of these appeared to contain RGD-623-Tamra. Early co-localization of RGD-623-Tamra with αᵥβ₃ was also observed. See FIG. 7B. At late (24 h) times some co-loacalization of RGD-623-Tamra with TG230, a marker for the trans-Golgi compartment, was observed. See FIG. 7C. No significant co-localization of RGD-623-Tamra with other markers (e.g., EAA1, LAMP1 and clathrin) was observed. While there is always concern about relying on fluorophore labels, the fact that these studies were performed using rather stable 2′-OMe phosphorothiate oligonucleotides makes it likely that the 3′-Tamra label probes a good indication of the subcellular distribution of the oligonucleotide.

In addition, the effects of some known inhibitors of endocytosis on the ability of cells to accumulate RGD-623-Tamra were examined. As seen in FIG. 7D, both β-cyclodextrin and cytochalasin D block the uptake of the RGD-oligonucleotide conjugate. At the non-cytotoxic concentrations used, β-cyclodextrin is thought to interfere with endocytosis mediated by lipid raft-rich structures, including caveolae, via depletion of cholesterol (see Parpal et al. (2001) J. Biol. Chem., 276, 9670-9678) while cytochalasin D blocks actin filament function (see Aplin and Juliano (1999) J. Cell Sci., 112, 695-706) which is necessary for almost all forms of endocytosis (see Kirkham and Parton (2005) Biochim. Biophys. Acta., 1745, 273-286). Without being bound to any one theory, the data suggests that the RGD-oligonucleotide conjugate enters cells via caveolae and possibly other lipid raft-rich smooth endocytotic vesicles.

### Example 8

### Toxicity

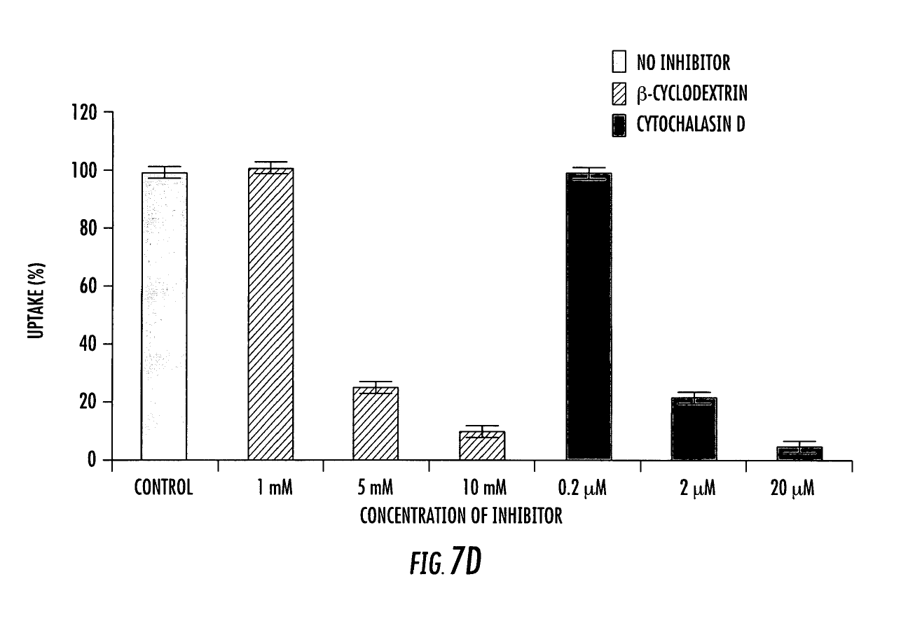

The short-term toxicity of the oligonucleotides and conjugates used in these studies was evaluated. As seen in FIG. 8, there was little effect of either the 623-Tamra oligonucleotide or its RGD-conjugate at concentrations up to 1000 nM. Some cell rounding was observed at the higher concentrations of RGD-oligonucleotide conjugate, but this did not result in significant cell loss. Although high concentrations of the RGD-conjugate might be expected to perturb adhesion mediated by RGD-binding integrins, in the experimental setting the cells have the opportunity to lay down extracellular matrix and may be anchored to that matrix by both RGD-binding and non-RGD-binding integrins, for example those involved in binding to collagen or laminin. See Juliano (2002) Annu. Rev. Pharmacol. Toxicol., 42, 283-323. Some toxicity was observed for the LIPOFECTAMINE™ 2000/623-Tamra complex at 100 nM oligonucleotide, while use of 200 nM was very toxic (not shown). Thus the RGD-oligonucleotide conjugate seems to be well-tolerated by cells over the concentration range needed to obtain significant effects in terms of splice correction of the luciferase reporter gene.

### Example 9

### General Methods for Albumin Conjugates

Human serum albumin (HSA) was purchased from Sigma-Aldrich (St. Louis, Mo., United States of America). Alexa Fluor 488 C5 maleimide and a CBQCA amino assay kit were obtained from Invitrogen (Carlsbad, Calif., United States of America). Dual-functionalized polyethylene glycol), Malhex-NH-PEG-O—C3H6-CONHS (MW 5000) was from Rapp Polymere (Tubingen, Germany). Cyclo-[RGDfK(Ac-SCH2CO)] peptide was purchased from Peptide International (Louisville, Ky., USA). Sulfosuccinimidyl 6-(3′-[2-pyridyldithio]-propionamido) hexanoate (Sulfo-LC-SPDP) was from Thermo Fisher Scientific (Rockford, Ill., United States of America). Glass-bottom tissue culture plates were obtained from MatTek (Ashland, Mass., United States of America) Oligonucleotides functionalized at their 5′ ends with thiol and in some cases at their 3′ ends with Tamra were prepared as described above in Example 1.

Cells: A375SM-Luc705-B cells were prepared as described above in Example 1 and were grown in Dulbecco's Modified Eagle's medium (DMEM; Invitrogen, Carlsbad, Calif., United States of America) supplemented with 10% fetal bovine serum (FBS).

Preparation of Albumin Conjugates with Cyclic RGD Peg or Cysteine PEG: Human serum albumin (HSA; 5 mg, 7.4×10⁻⁸ mol) was reacted with either L-cysteine (0.05 mg, 3.67×10⁻⁷ mol) or Alexa Fluor 488 C5 maleimide (0.27 mg, 3.67×10⁻⁷ mol) in phosphate buffered saline (PBS) supplemented with 1 nM EDTA (pH 7.4) for 4 h at room temperature to conjugate the single surface thio group on albumin. The reaction mixture was dialyzed (MW cutoff 3500). The amino groups of the albumin were then reacted with Malhex-NH-PEG-O—C3H6-CONHS (11 mg, 2.21×10⁻⁶ mol) in PBS/1 mM EDTA (pH 7.4) for 4 h at room temperature. The product, a human albumin derivative (PA) with PEG-maleimide groups on the surface was purified from unincorporated PEG materials by dialysis (MW cutoff 100,000). The average number of PEG groups conjugated to albumin was determined by using the CBQCA assay (Molecular Probes, Eugene, Oreg., United States of America), according to the manufacturer's instruction, to measure residual free amino groups. The thiol group on cyclic RGDfK needed for conjugation with the maleimidie group on albumin was freshly generated by 1 h incubation of cyclo-[RGDfK(Ac-SCH₂CO)] (5 mg, 7.35×10⁻⁶ mol) in pH 7.0 deprotection buffer (HEPES (50 mM)), NH₂OH (50 mM) and EDTA (30 mM) at room temperature. The maleimide groups on the albumin derivative were then reacted with the thiol group of cyclic RGDfK (or cysteine as control) in PBS with 1 mM EDTA (pH 7.4) overnight at room temperature and purified by dialysis (MW cutoff 100,000).

Conjugation of RGD-PEG-Albumin with Oligonucleotides: PEG-albumin conjugates, derivatized with either cyclo RGD (i.e., the intermediate conjugate termed RPA; 9.2 mg, 7.35×10⁻⁸ mol) or cysteine (i.e., the intermediate conjugate termed CPA; 8.7 mg, 7.35×10⁻⁸ mol) were reacted with dual functional sulfosuccinimidyl 6-(3′-[2-pyridyldithio]propionamido)hexanoate (1.2 mg, 2.21×10⁻⁶ mol) linker in PBS with 1 mM EDTA (pH 7.4) for 4 h at room temperature and purified by dialysis (MW cutoff 100,000). Thiol-derivatized 623 oligonucleotide (or a mismatch version, 5MM623-SH) was then added to the intermediate in PBS with 1 mM EDTA (pH 7.4), and the reaction was maintained at room temperature for 24 h and purified by dialysis (MW cutoff 100,000). The average number of oligonucleotides linked to albumin was determined as 9.8 by observing the release of pyridine-2-thione (Aₘₐₓ=343 nm) from the reaction intermediate. This was also confirmed by monitoring the increase in OD260 subsequent to the oligonucleotide conjugation when the reaction was followed by UV spectroscopy.

Physical Characterization of the RGD-PEG-Oligonucleotide Albumin Conjugates: The cyclic RGD derivatized albumin conjugate of oligonucleotide (RPAO) was analyzed by gel filtration fast protein liquid chromatography (FPLC) using a Superose™ 6 10/300 size exclusion column (GE Healthcare, Chalfont St. Giles, United Kingdom). The size and polydispersity of the conjugates were determined by a quasi-elastic dynamic light scattering (QELS) method as they eluted from the column.

Oligonucleotide Treatment of Cells and Luciferase Assay: A375SM-Luc705-B cells were seeded onto 12 well plates at 1×10⁵ cells per well in medium containing 10% FBS. After 24 h, cells were rinsed, placed in OPTI-MEM (Invitrogen, Carlsbad, Calif., United States of America) and treated with free 623 oligonucleotide, various versions of the albumin-PEG-623 oligonucleotide conjugates, or 623 oligonucleotide complexed with LIPOFECTAMINE™ 2000 (Invitrogen, Carlsbad, Calif., United States of America) per manufacturer's instruction. After 4 h of treatment, FBS was added to each well to 1%. After 24 h, cells were washed with DMEM medium containing 10% FBS and incubated in DMEM supplemented with 1% FBS for 48 h prior to harvest. Activation of luciferase gene expression due to correction of splicing by oligonucleotide 623 was determined using a Luciferase assay kit (Promega, Madison, Wis., United States of America) on a Monolight 2010 instrument (Analytical Luminescence Laboratory, San Diego, Calif., United States of America)

Cellular Uptake and Confocal Microscopy: Cells were seeded onto either 12 well plates at 1×10⁵ cells per well for cellular uptake measurements or in 12 well glass-bottom plates at 2×10⁴ cells per well for live cell analysis by confocal microscopy. After treatment of cells with Tamra labeled oligonucleotide derivatives for 24 h, the cells were either lysed for measurement of Tamra fluorescence using a Nanodrop microfluorimeter (Nanodrop Technologies, Wilmington, Deleware, United States of America) or washed with DMEM containing 10% FBS and then placed in DMEM without phenol red, supplemented with 1% FBS, for confocal microscopy analysis. Co-localization of Tamra-labeled oligonucleotides with Alexa 488 labeled Transferrin or Dextran (Molecular Probes, Beaverton, Oreg., United States of America) in live cell was also examined by confocal microscopy. An Olympus confocal microscope with a 60× objective lens was used, and the data were processed using Fluoview software (Olympus, Center Valley, Pa., United States of America) as described in Example 1.

Toxicity Studies: Cells were treated with various concentrations of oligonucleotides or conjugates. After 48 h of treatment, cells were trypsinized and viable cells were counted using an electric particle counter for short-term toxicity studies or replated at low density in 6 well plates containing a mixture of 1% low gelling temperature agarose (SeaKem, Rockland, Me., United States of America) supplemented with DMEM-H/10% FBS for long-term (14 day) studies of colony forming ability.

Nuclease Stability: Micrococcal nuclease (4000 gel units) was added to solutions of 623-Tamra or RPA-623-Tamra and the samples were incubated for various periods of time at 37° C. The reactions were then stopped by the addition of EDTA (50 mM), and the fluorescent samples were analyzed on 10% polyacrylamide gels and examined under long-wavelength ultraviolet illumination to detect possible nucleolytic cleavage of the Tamra-labeled oligonucleotides.

### Example 10

### Synthesis and Characterization of Albumin-PEG-Antisense Oligonucleotide Conjugates

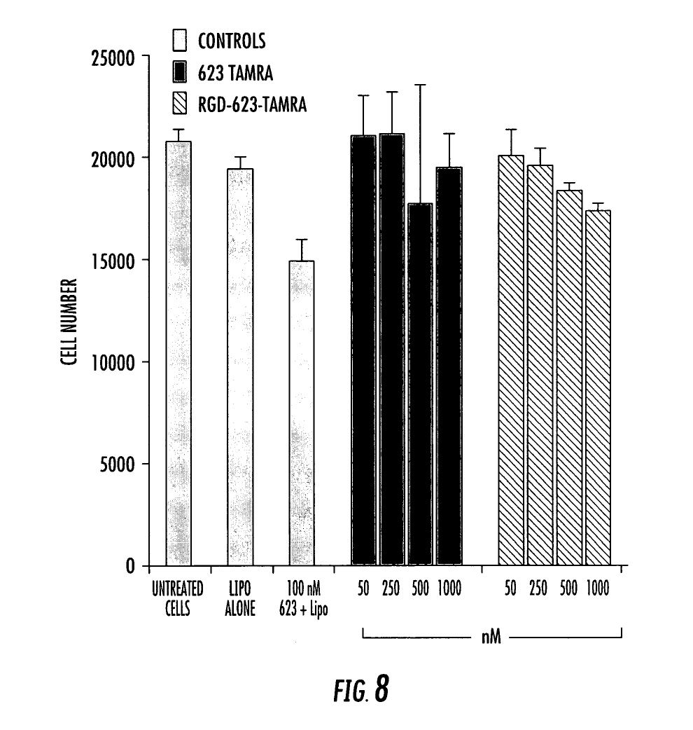

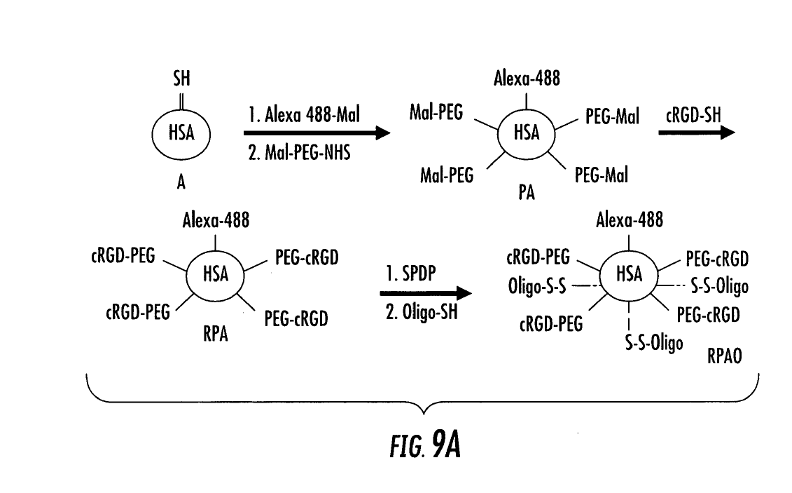

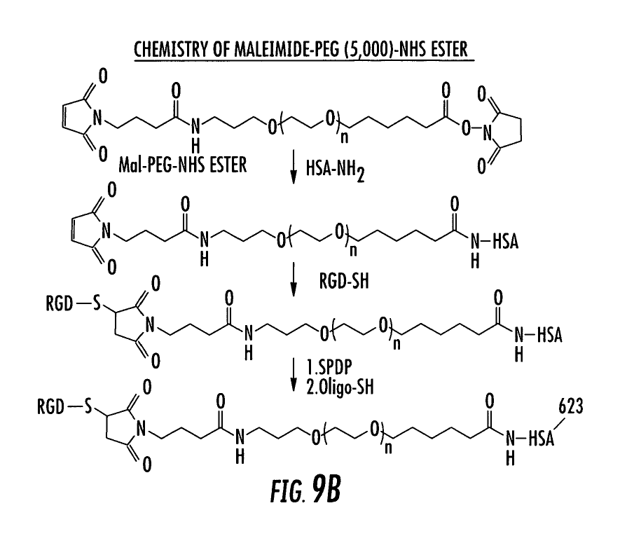

The overall strategy for the preparation of the albumin conjugates is outlined in FIGS. 9A and 9B. First, the single sulfhydryl group on human serum albumin was labeled with the green fluorophore Alexa 488 (alternatively, the sulfhydryl can be blocked by forming an S—S bridge with cysteine). Subsequently, several surface amino groups were reacted with MaI-Peg-NHS to form a pegylated albumin (PA). The number of PEG chains conjugated to PA was determined using a CBQCA assay to quantitate the number of exposed amino groups on albumin before and after the reaction with PEG. In the PA intermediate prepared according to Example 9, CBQCA assay indicated 10 PEG chains had been conjugated per albumin molecule. After purification of the PA conjugate, excess thiol-containing cyclic RGD peptide was reacted with the terminal maleimide groups on PA to form RGD-PEG-albumin (RPA). As a control, cysteine was used instead of RGD to react with PA to form Cys-PEG-albumin (CPA). After purification, additional exposed amino groups were reacted with the bifunctional reagent Sulfo-LC-SPDP, and then, the 623-SH oligonucleotide (or a mismatch version, 5MM623-SH) was conjugated to form RPA-Oligonucleotide (RPAO). In some cases, the 623-SH included a 3′-Tamra (red) fluorophore (with the resulting product termed RPA-623-Tamra).

The number of oligonucleotides linked to the conjugate was determined in two ways. First, formation of the colored product pyridine-2-thione (λₘₐₓ=343 nm) was monitored as the 5′-thiol oligonucleotide reacted with the SPDP-conjugated albumin. See FIG. 10. Panel A of FIG. 10 shows the UV spectra of RGD-PEG-albumin. The peak labeled 1 is for Alexa 488. Panel B of FIG. 10 shows the reaction mixture of 623-SH and RGD-PEG-albumin that had been reacted with Sulfo-LC-SPDP. As in Panel A, the peak labeled 1 is for Alexa 488. The peak labeled 2 is for pyridine-2-thione, and the peak labeled 3 is for 623. Panel C of FIG. 10 shows the UV spectra of the oligonucleotide-RGD-albumin conjugate, where the peak labeled 1 is for Alexa 488, the peak labeled 2 is for pyridine-2-thione, and the peak labeled 3 is for 623. Second, the OD260 was determined before and after the conjugation reaction. Both of these methods led to close agreement with 8-11 oligonucleotides linked per albumin in various preparations.

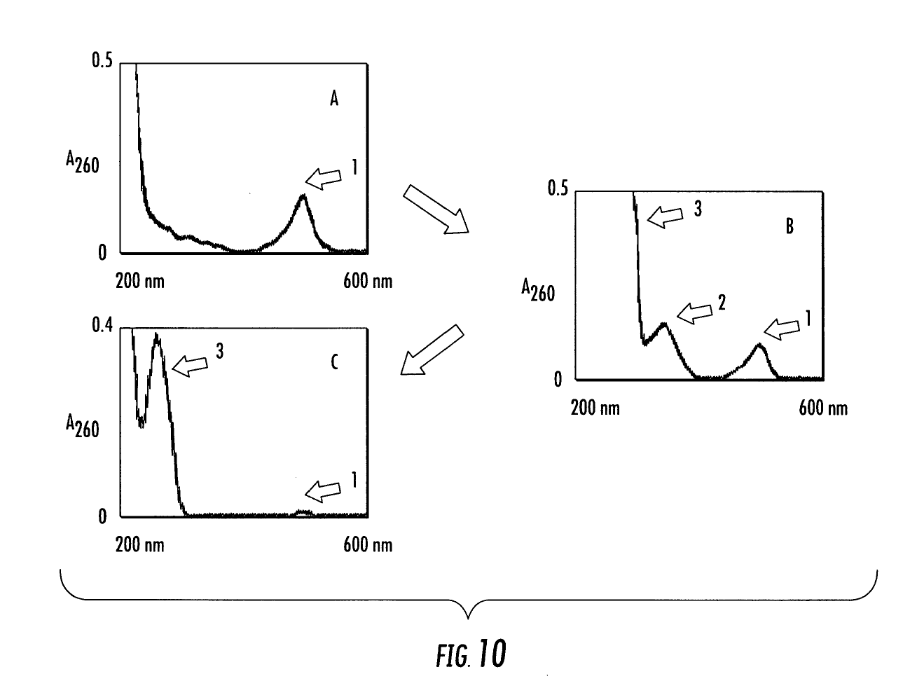

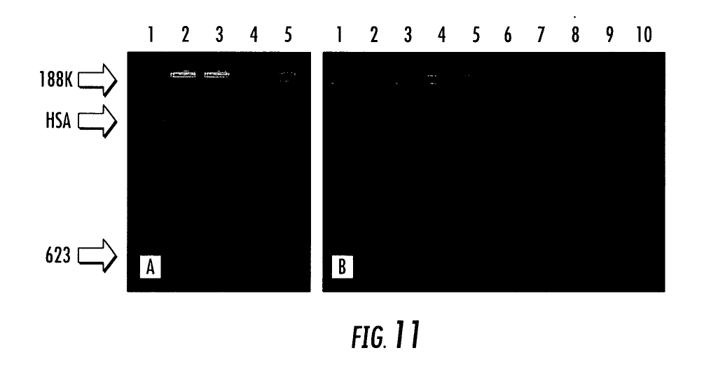

The polyacrylamide gel migration behavior of the starting materials and the conjugates is illustrated in panel A of FIG. 11. The 623-Tamra and RPA-623-Tamra are detected by their red fluorescence, while the RPA is detected by its green fluorescence (Alexa 488). As seen, Alexa 488-modified HSA migrated well into the gel, consistent with its molecular weight of 68 kDa, while the unconjugated 623-Tamra oligonucleotide migrated near the dye front. Both RPA and RPA-623-Tamra failed to significantly enter the gel, indicating molecular size greater than the largest molecular weight marker used (188 kDa). Gel analysis was also used to evaluate the related nuclease stability of 623-Tamra versus that of RPA-623-Tamra. As seen in panel B of FIG. 11, although 623-Tamra is a rather stable 2′-O-Me-phosphorothioate oligonucleotide, incubation with micrococcal nuclease caused a gradual degradation of this material. In contrast, there was no loss of Tamra-labeled oligonucleotide from the RPA-623-Tamra conjugate, suggesting that the olignucleotides linked to PEG-albumin are partially protected against nuclease degradation.

The molecular size of the RPA-623 conjugate was estimated using size-exclusino chromatography and quasi-elastic laser light scattering. As indicated in FIG. 12, the RPA-623 conjugate is heterodisperse, migrating as a broad peak while albumin has a sharper migration profile. The hydrodynamic radius of the RPA-623 conjugate was estimated at approximately 6 nm, while that of albumin was estimated at 2.2 nm. Thus, the average radius of the conjugate is about 2.7 times that of the unmodified albumin carrier. The albumin-oligonucleotide conjugates were stable during several weeks of storage in buffer at 4° C., with no indication of aggregation.

### Example 11

### Pharmacological Effect of Targeted Albumin-Oligonucleotide Conjugates

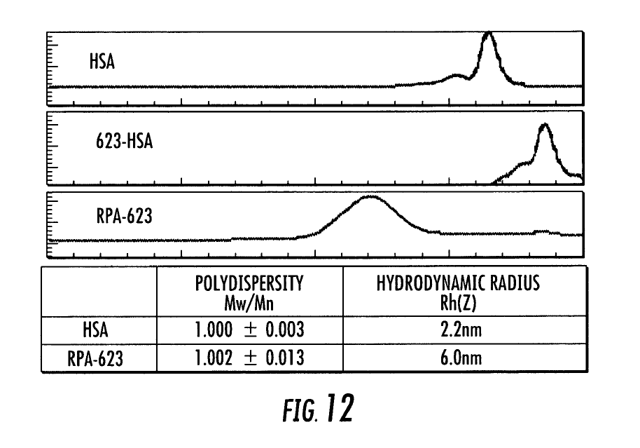

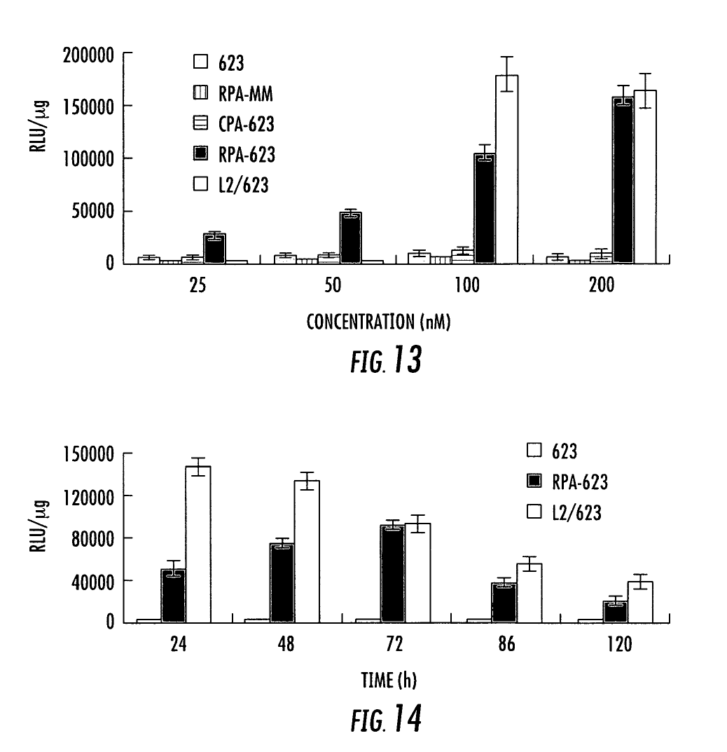

To evaluate the pharmacological effectiveness of the albumin conjugates, A375SM-Luc705-B cells were incubated with various concentrations of free antisense oligonucleotide 623 (SEQ ID NO: 1), 623 oligonucleotide complexed with the cationic lipid LIPOFECTAMINE™ 2000 (Invitrogen, Carlsbad, Calif., United States of America), or different versions of albumin conjugate. As illustrated in FIG. 13, treatment with the RPA-623 conjugate resulted in a concentration-dependent increase in luciferase activity over the range of 25-200 nM, while use of a mismatched oligonucleotide version (RPA-MM), or a version where the PEG chains were terminated with cysteine (CPA-623), failed to achieve an effect. “Free” 623 oligonucleotide (SEQ ID NO: 1) did not cause a significant increase in luciferase activity.

The magnitude of the effect achieved by the RPA-623 conjugate almost equaled that of the LIPOFECTAMINE™ complex, which is often considered the “gold standard” for delivery of oligonucleotides to cells in culture. However, the time course of luciferase activation differed markedly between RPA-623 conjugate and the 623 LIPOFECTAMINE™ complex. Thus, as seen in FIG. 14, treatment with RPA-623 resulted in activity that rose gradually, attained a maximum at 72 h post-treatment, and then gradually declined. In contrast, use of the 623-LIPOFECTAMINE™ complex resulted in a rapid rise of activity within 24 h of treatment followed by a monotonic decline. This pattern suggests that the LIPOFECTAMINE™ complex rapidly delivers oligonucleotide to the cytosol and nucleus, while delivery of oligonucleotide from the RPA-623 conjugate involves the αvβ3 integrin since, as seen in FIG. 15, incubation with excess cyclic RGD peptide completely blocks the ability of the conjugate to activate luciferase. At the concentrations used, the RGD peptide does not cause cell detachment or other obvious toxicity.

### Example 12

### Cellular Uptake and Confocal Microscopy

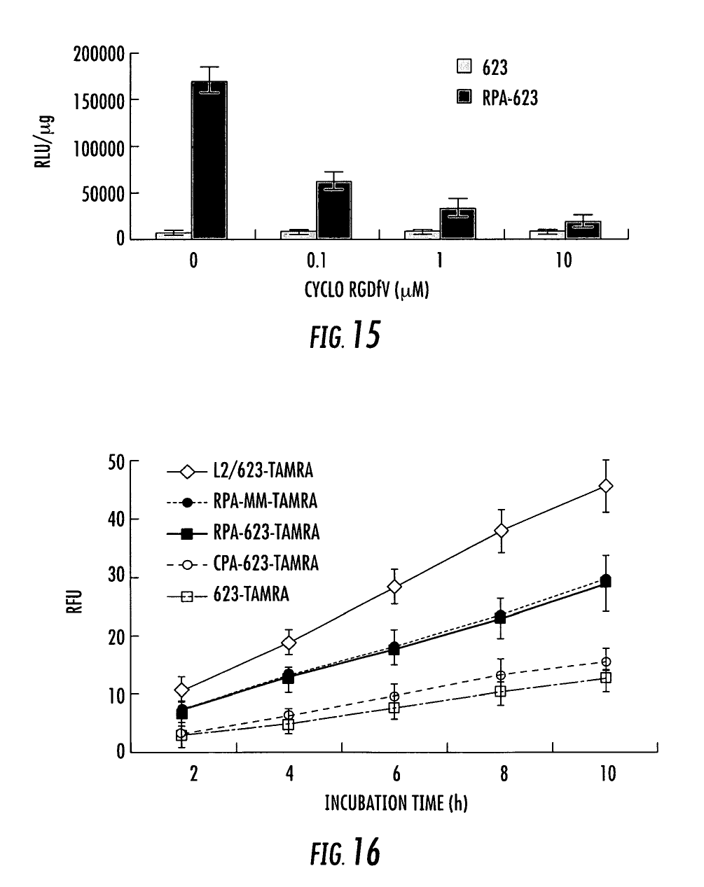

Total cellular accumulation of the various conjugates was measured using a fluorimeter assay. As shown in FIG. 16, uptake was approximately linear with time in all cases. The highest uptake was observed with 623-Tamra complexed with LIPOFECTAMINE™ 2000, followed by RPAO (with either 623 oligonucleotide or mixmatched oligonucleotide), followed by CPA-623 and free 623-Tamra.

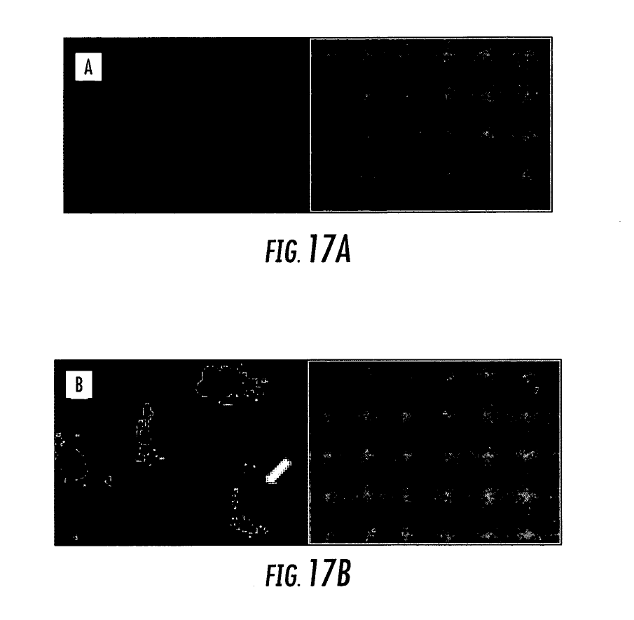

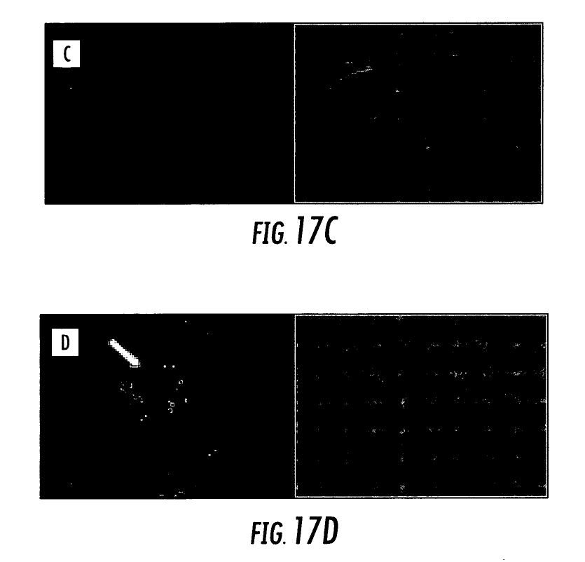

The subcellular distribution of the 623-Tamra labeled oligonucleotide was also studied after delivery of the conjugates or after delivery of the LIPOFECTAMINE complexes. As seen in FIGS. 17B and 17D, live cells treated with either RPA-623-Tamra or a LIPOFECTAMINE™/623-Tamra complex displayed substantial intracellular Tamra fluorescence at 24 h, including material present in cytoplamic vesicles as well as in the nucleus. By contrast, cells treated with free 623 Tamra or with CPA-623-Tamra exhibited less intracellular fluorescene with no evidence of nuclear accumulation. See FIGS. 17A and 17C. This data suggests that a RGD-PEG-albumin conjugate can provide effective delivery of pendant oligonucleotides to the cell nucleus.

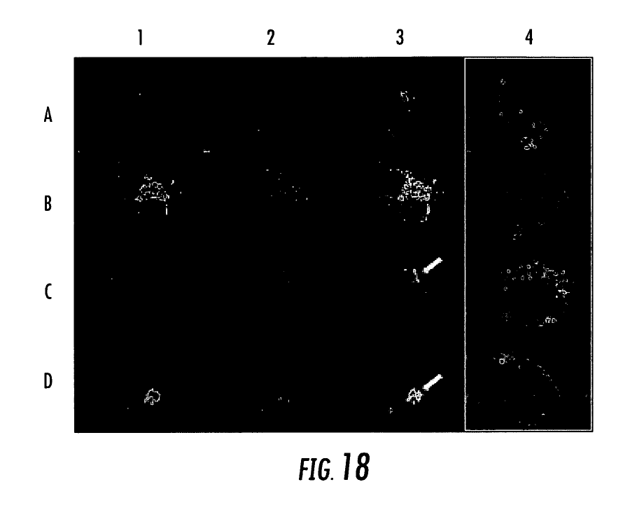

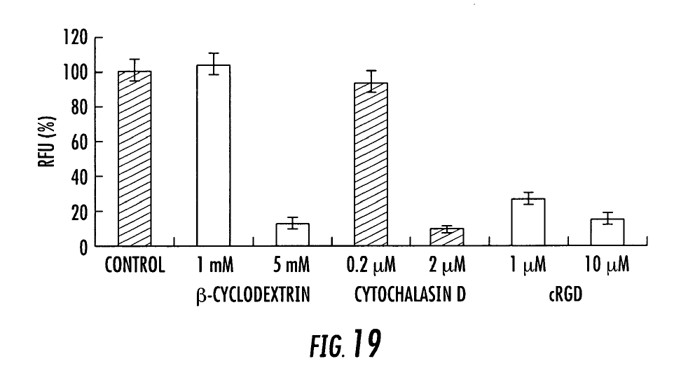

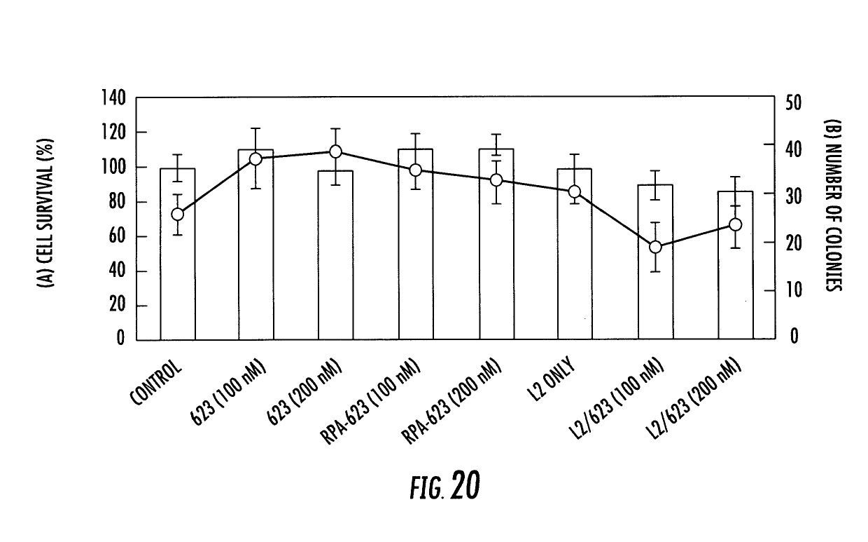

To further understand the cellular uptake and trafficking of the conjugates, the conjugates were co-incubated with well-known markers for different endocytotic pathways and their subcellular distributions were compared. Transferrin was used as a marker for clathrin-coated vesicle-mediated uptake and dextran was used as a marker for smooth vesicle endocytosis. See Kirkham and Parton (2005) Biochim. Biophys. Acta., 1745, 273-286. Transferrin and dextran were labeled with Alexa 488, a green fluorophore, while the RPA-623-Tamra conjugate displays a red fluorescence. As seen in FIG. 18, at early time points (2 h), there was little overlap of the RPA-623-Tamra fluorescence with that of transferrin (FIG. 18, row A, column 3), but there was substantial overlap with the dextrin fluorescence (FIG. 18, row C, column 3). At 24 h, there was fluorescence overlap in both cases (FIG. 18, rows B and D, column 3), although it was most pronounced for dextran. Nuclear accumulation of Tamra fluorescence was observed at the later time points, similar to that seen in FIG. 17. These observations suggest that the RGD-PEG-albumin-oligonucleotide conjugate (RPAO) is initially taken up via smooth vesicle endocytosis, but later the material enters endomembrane compartments that are accessible via both the coated vesicle and smooth vesicle pathways. Inhibitor studies were consistent with this interpretation. Thus, as seen in FIG. 19, cellular accumulation of the RPA-623-Tamra was inhibited by nontoxic concentrations of β-cyclodextrin and cytochalasin D (as well as by excess RGD peptide). This indicates that the RPA-623-Tamra is taken up by an actin-dependent pathway that involves smooth vesicles rich in lipid-raft components.

### Example 13

### Toxicity Studies

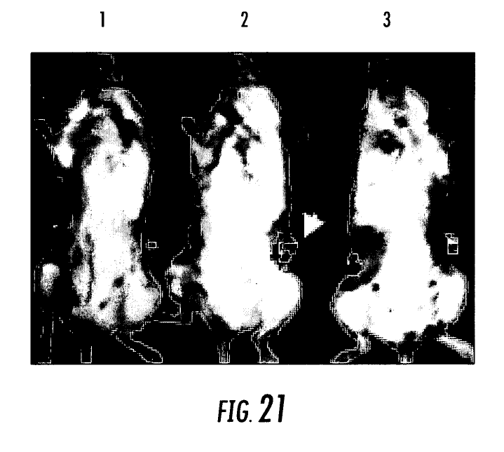

Both the short-term and long-term toxicity of the albumin conjugates were examined as described in Example 9. As seen in FIG. 20, there was little acute or long-term toxicity of RPA-623 conjugate, even when used at concentrations needed to obtain a strong pharmacological effect.

### Example 14

### In Vivo Targeting of RGD-623 Oligonucleotide

Immunodeficient mice (SCID mice) were inoculated with A375 melanoma cells containing an inducible luciferase reporter gene in the right flank. The reporter gene contains an abnormal intron inserted into its coding region and, thus, does not code for luciferase protein. However, upon effective delivery of splice shifting antisense oligonucleotide to the cell nucleus, the abnormal intron is spliced out and luciferase protein is produced. Therefore the reporter gene provides an effective readout for delivery of a SSO to a tumor in vivo.

The tumors were allowed to grow to a certain size and then the mice were injected with either saline; RGD-623; or free 623 oligonucleotide (SEQ ID NO: 1). After a period of incubation to allow the SSO to act, the mice were injected with luciferin and the activity of the luciferase in the tumors was monitored by bioluminescence imaging using an IVIS imaging system. FIG. 21 shows that the tumor in the mouse (2) that received RGD-623 showed a substantial increase in luciferase activity compared to the mice that recieved saline (1) or free oligonucleotide (3).

### REFERENCES

The references listed below, as well as all references cited in the specification, are incorporated herein by reference to the extent that they supplement, explain, provide a background for, or teach methodology, techniques, and/or compositions employed herein.
Abes, S., Moulton, H., Turner, J., Clair, P., Richard, J. P., Iversen, P., Gait, M. J. and Lebleu, B. (2007) Peptide-based delivery of nucleic acids: design, mechanism of uptake and applications to splice-correcting oligonucleotides. Biochem Soc Trans, 35, 53-55.
Abes, S., Turner, J. J., Ivanova, G. D., Owen, D., Williams, D., Arzumanov, A., Clair, P., Gait, M. J. and Lebleu, B. (2007) Efficient splicing correction by PNA conjugation to an R6-Penetratin delivery peptide. Nucleic acids research, 35, 4495-4502.
Aplin, A. E., and Juliano, R. L. (1999) Integrin and cytoskeletal regulation of growth factor signaling to the MAP kinase pathway. J. Cell Sci., 112 (Pt 5), 695-706.
Astriab-Fisher, A., Fisher, M. H., Juliano, R. and Herdewijn, P. (2004) Increased uptake of antisense oligonucleotides by delivery as double stranded complexes. Biochem Pharmacol, 68, 403-407.
Astriab-Fisher, A., Sergueev, D. S., Fisher, M., Shaw, B. R. and Juliano, R. L. (2000) Antisense inhibition of P-glycoprotein expression using peptide-oligonucleotide conjugates. Biochem Pharmacol, 60, 83-90.
Astriab-Fisher, A., Sergueev, D., Fisher, M., Shaw, B. R. and Juliano, R. L. (2002) Conjugates of antisense oligonucleotides with the Tat and antennapedia cell-penetrating peptides: effects on cellular uptake, binding to target sequences, and biologic actions. Pharm Res, 19, 744-754.
Bendifallah, N., Rasmussen, F. W., Zachar, V., Ebbesen, P., Nielsen, P. E. and Koppelhus, U. (2006) Evaluation of cell-penetrating peptides (CPPs) as vehicles for intracellular delivery of antisense peptide nucleic acid (PNA). Bioconjug Chem, 17, 750-758.
Caswell, P. T. and Norman, J. C. (2006) Integrin trafficking and the control of cell migration. Traffic (Copenhagen, Denmark), 7, 14-21.
Chen, X., Plasencia, C., Hou, Y. and Neamati, N. (2005) Synthesis and biological evaluation of dimeric RGD peptide-paclitaxel conjugate as a model for integrin-targeted drug delivery. J Med Chem, 48, 1098-1106.
Chiu, Y. L., Ali, A., Chu, C. Y., Cao, H. and Rana, T. M. (2004) Visualizing a correlation between siRNA localization, cellular uptake, and RNAi in living cells. Chem Biol, 11, 1165-1175.
Coppelli, F. M. and Grandis, J. R. (2005) Oligonucleotides as anticancer agents: from the benchside to the clinic and beyond. Curr Pharm Des, 11, 2825-2840.
Crooke, S. T. (2004) Progress in antisense technology. Annu Rev Med, 55, 61-95.
El-Andaloussi, S., Johansson, H. J., Lundberg, P. and Langel, U. (2006) Induction of splice correction by cell-penetrating peptide nucleic acids. The journal of gene medicine, 8, 1262-1273.
Fattal, E., Couvreur, P. and Dubernet, C. (2004) “Smart” delivery of antisense oligonucleotides by anionic pH-sensitive liposomes. Adv Drug Deliv Rev, 56, 931-946.
Fisher, M., Abramov, M., Van Aerschot, A., Xu, D., Juliano, R. L. and Herdewijn, P. (2007) Inhibition of MDR1 expression with altritol-modified siRNAs. Nucleic Acids Res, 35, 1064-1074.
Friedlander, M., Theesfeld, C. L., Sugita, M., Fruttiger, M., Thomas, M. A., Chang, S, and Cheresh, D. A. (1996) Involvement of integrins alpha v beta 3 and alpha v beta 5 in ocular neovascular diseases. Proceedings of the National Academy of Sciences of the United States of America, 93, 9764-9769.
Hoekstra, D., Rejman, J., Wasungu, L., Shi, F. and Zuhorn, I. (2007) Gene delivery by cationic lipids: in and out of an endosome. Biochemical Society transactions, 35, 68-71.
Hu-Lieskovan, S., Heidel, J. D., Bartlett, D. W., Davis, M. E. and Triche, T. J. (2005) Sequence-specific knockdown of EWS-FLI1 by targeted, nonviral delivery of small interfering RNA inhibits tumor growth in a murine model of metastatic Ewing's sarcoma. Cancer research, 65, 8984-8992.
Inoue, A., Sawata, S. Y. and Taira, K. (2006) Molecular design and delivery of siRNA. J Drug Target, 14, 448-455.
Jarver, P. and Langel, U. (2004) The use of cell-penetrating peptides as a tool for gene regulation. Drug Discov Today, 9, 395-402.
Juliano, R. L. and Yoo, H. (2000) Aspects of the transport and delivery of antisense oligonucleotides. Curr Opin Mol Ther, 2, 297-303.
Juliano, R. L. (2002) Signal transduction by cell adhesion receptors and the cytoskeleton: functions of integrins, cadherins, selectins, and immunoglobulin-superfamily members. Annu Rev Pharmacol Toxicol, 42, 283-323.
Juliano, R. L. (2005) Peptide-oligonucleotide conjugates for the delivery of antisense and siRNA. Curr Opin Mol Ther, 7, 132-136.
Juliano, R. L. (2007) In Mirkin, C. and Niemeyer, C. (eds.), Nanobiotechnology. Wiley and Sons, New York, Vol. 2.
Kang, S. H., Cho, M. J. and Kole, R. (1998) Up-regulation of luciferase gene expression with antisense oligonucleotides: implications and applications in functional assay development. Biochemistry, 37, 6235-6239.
Kim, D. H. and Rossi, J. J. (2007) Strategies for silencing human disease using RNA interference. Nat Rev Genet, 8, 173-184.
Kirkham, M., and Parton, R. G. (2005) Clathrin-independent endocytosis: new insights into caveolae and non-caveolar lipid raft carriers. Biochim. Biophys. Acta., 1745, 273-286.
Kole, R., Vacek, M. and Williams, T. (2004) Modification of alternative splicing by antisense therapeutics. Oligonucleotides, 14, 65-74.
Kurreck, J. (2003) Antisense technologies. Improvement through novel chemical modifications. Eur J Biochem, 270, 1628-1644.
Lundberg, P., El-Andaloussi, S., Sutlu, T., Johansson, H. and Langel, U. (2007) Delivery of short interfering RNA using endosomolytic cell-penetrating peptides. Faseb J.
Lv, H., Zhang, S., Wang, B., Cui, S, and Yan, J. (2006) Toxicity of cationic lipids and cationic polymers in gene delivery. J Control Release, 114, 100-109.
Manoharan, M. (2002) Oligonucleotide conjugates as potential antisense drugs with improved uptake, biodistribution, targeted delivery, and mechanism of action. Antisense Nucleic Acid Drug Dev, 12, 103-128.
McCaffrey, A. P., Meuse, L., Pham, T. T., Conklin, D. S., Hannon, G. J. and Kay, M. A. (2002) RNA interference in adult mice. Nature, 418, 38-39.
Mitra, A., Coleman, T., Borgman, M., Nan, A., Ghandehari, H. and Line, B. R. (2006) Polymeric conjugates of mono- and bi-cyclic alphaVbeta3 binding peptides for tumor targeting. J Control Release, 114, 175-183.
Moulton, H. M., Hase, M. C., Smith, K. M. and Iversen, P. L. (2003) HIV Tat peptide enhances cellular delivery of antisense morpholino oligomers. Antisense Nucleic Acid Drug Dev, 13, 31-43.
Muratovska, A. and Eccles, M. R. (2004) Conjugate for efficient delivery of short interfering RNA (siRNA) into mammalian cells. FEBS Lett, 558, 63-68.
Oishi, M., Hayama, T., Akiyama, Y., Takae, S., Harada, A., Yamasaki, Y., Nagatsugi, F., Sasaki, S., Nagasaki, Y. and Kataoka, K. (2005) Supramolecular assemblies for the cytoplasmic delivery of antisense oligodeoxynucleotide: polyion complex (PIC) micelles based on poly(ethylene glycol)-SS-oligodeoxynucleotide conjugate. Biomacromolecules, 6, 2449-2454.
Parpal, S., Karlsson, M., Thorn, H., and Stralfors, P. (2001) Cholesterol depletion disrupts caveolae and insulin receptor signaling for metabolic control via insulin receptor substrate-1, but not for mitogen-activated protein kinase control. J. Biol. Chem., 276, 9670-9678.
Perret, E., Lakkaraju, A., Deborde, S., Schreiner, R. and Rodriguez-Boulan, E. (2005) Evolving endosomes: how many varieties and why? Current opinion in cell biology, 17, 423-434.
Resina, S., Kole, R., Travo, A., Lebleu, B. and Thierry, A. R. (2007) Switching on transgene expression by correcting aberrant splicing using multi-targeting steric-blocking oligonucleotides. J Gene Med, 9, 498-510.
Soutschek, J., Akinc, A., Bramlage, B., Charisse, K., Constien, R., Donoghue, M., Elbashir, S., Geick, A., Hadwiger, P., Harborth, J. et al. (2004) Therapeutic silencing of an endogenous gene by systemic administration of modified siRNAs. Nature, 432, 173-178.
Stupack, D. G. and Cheresh, D. A. (2004) Integrins and angiogenesis. Curr Top Dev Biol, 64, 207-238.
Turner, J. J., Arzumanov, A. A. and Gait, M. J. (2005) Synthesis, cellular uptake and HIV-1 Tat-dependent trans-activation inhibition activity of oligonucleotide analogues disulphideconjugated to cell-penetrating peptides. Nucleic acids research, 33, 27-42.
Turner, J. J., Ivanova, G. D., Verbeure, B., Williams, D., Arzumanov, A. A., Abes, S., Lebleu, B. and Gait, M. J. (2005) Cell-penetrating peptide conjugates of peptide nucleic acids (PNA) as inhibitors of HIV-1 Tat-dependent trans-activation in cells. Nucleic Acids Res, 33, 6837-6849.
Van den Haute, C., Eggermont, K., Nuttin, B., Debyser, Z. and Baekelandt, V. (2003) Lentiviral vector-mediated delivery of short hairpin RNA results in persistent knockdown of gene expression in mouse brain. Hum Gene Ther, 14, 1799-1807.
Vinogradov, S. V., Batrakova, E. V. and Kabanov, A. V. (2004) Nanogels for oligonucleotide delivery to the brain. Bioconjug Chem, 15, 50-60.
Wadia, J. S. and Dowdy, S. F. (2002) Protein transduction technology. Curr Opin Biotechnol, 13, 52-56.
White, D. P., Caswell, P. T. and Norman, J. C. (2007) alpha v beta3 and alpha5beta1 integrin recycling pathways dictate downstream Rho kinase signaling to regulate persistent cell migration. The Journal of cell biology, 177, 515-525.
Xu, D., McCarty, D., Fernandes, A., Fisher, M., Samulski, R. J. and Juliano, R. L. (2005) Delivery of MDR1 small interfering RNA by self-complementary recombinant adenoassociated virus vector. Mol Ther, 11, 523-530.
Yoo, H. and Juliano, R. L. (2000) Enhanced delivery of antisense oligonucleotides with fluorophore-conjugated PAMAM dendrimers. Nucleic Acids Res, 28, 4225-4231.

It will be understood that various details of the presently disclosed subject matter can be changed without departing from the scope of the presently disclosed subject matter. Furthermore, the foregoing description is for the purpose of illustration only, and not for the purpose of limitation.

1. A composition for delivering an oligonucleotide to a target cell through endocytosis, wherein the composition comprises a conjugate comprising one or more ligand groups capable of mediating receptor endocytosis, one or more oligonucleotide groups each comprising an oligonucleotide, and a carrier macromolecule, wherein each of the ligand groups and each of the oligonucleotide groups are attached to the carrier macromolecule.

2. The composition of claim 1, wherein the oligonucleotide comprises one of the group consisting of an antisense RNA, a small interfering RNA (siRNA), and a micro RNA (miRNA) that selectively binds to an RNA in the target cell.

3. The composition of claim 2, wherein one or more oligonucleotide groups further comprise a detectable tag.

4. The composition of claim 3, wherein the detectable tag is attached to a 3′ end of the oligonucleotide.

5. The composition of claim 3, wherein the detectable tag is a fluorophore.

6. The composition of claim 5, wherein the detectable tag is a Tamra fluor.

7. The composition of claim 1, wherein the oligonucleotide is a therapeutic agent and the composition comprises a therapeutically effective amount of the oligonucleotide.

8. The composition of claim 1, wherein the composition is prepared for administration to a vertebrate subject.

9. The composition of claim 8, wherein the composition is prepared as a pharmaceutical formulation for administration to a mammalian subject.

10. The composition of claim 1, wherein the one or more ligand groups each comprise one or more peptide ligand.

11. The composition of claim 10, wherein the peptide ligand comprises a cyclic RGD peptide.

12. The composition of claim 1, wherein one or more ligand group comprises a combination of different types of ligands.

13. The composition of claim 12, wherein the different types of ligands are selected from the group consisting of EGF, CXCL12, CCL3, small organic molecule ligands for chemokine receptors, small organic molecule ligands for the P2Y subfamily of GPCR receptors, small organic molecule ligands for alpha and beta adrenergic receptors, terbutaline, phenylephrine, and peptide, peptidomimetic and non-peptide ligands for integrins including a5b1, a4b1 and LFA-1.

14. (canceled)

15. The composition of claim 1, wherein the carrier macromolecule is a protein.

16. The composition of claim 15, wherein the carrier macromolecule is a serum albumin protein.

17. The composition of claim 16, wherein the carrier macromolecule is human serum albumin.

18. The composition of claim 15, wherein one or more ligand group comprises a polyethylene glycol (PEG) moiety, wherein the PEG moiety is attached to the protein.

19. The composition of claim 18, wherein the PEG moiety is attached to the protein through an amide linkage.

20. The composition of claim 18, wherein one or more ligand group comprises a cyclic RGD peptide attached to the PEG group through a maleimide group.

21. The composition of claim 1 , wherein the one or more oligonucleotide groups are attached to the carrier macromolecule through an alkylene linker group.

22. The composition of claim 21, wherein the alkylene linker group is —S—(CH₂)₆—.

23-31. (canceled)

32. A method of delivering an oligonucleotide to a target cell through endocytosis, comprising contacting the cell with the composition of claim 1, wherein the oligonucleotide is actively transported into the target cell, wherein the target cell comprises one or more receptors capable of mediating receptor endocytosis in response to the one or more ligand groups.

33. The method of claim 32, wherein the target cell is present in a subject, and contacting the target cell with the composition comprises administering a therapeutically effective amount of the composition to the subject.

34. The method of claim 32, wherein the oligonucleotide comprises one of the group consisting of an antisense RNA, a small interfering RNA (siRNA), and a micro RNA (miRNA) that selectively binds to an RNA in a target cell.

35. The method of claim 32, wherein the one or more ligand groups each comprise a peptide ligand.

36. The method of claim 35, wherein one or more ligand groups comprise a combination of different types of ligands.

37. The method of claim 36, wherein the different types of ligands are selected from the group consisting of EGF, CXCL12, CCL3, small organic molecule ligands for chemokine receptors, small organic molecule ligands for the P2Y subfamily of GPCR receptors, small organic molecule ligands for alpha and beta adrenergic receptors, terbutaline, phenylephrine, and peptide, peptidomimetic and non-peptide ligands for integrins including a5b1, a4b1 and LFA-1.

38. (canceled)

39. The method of claim 32, wherein the carrier macromolecule is a protein.

40. The method of claim 39, wherein the carrier macromolecule is a serum albumin protein.

41. The method of claim 40, wherein the carrier macromolecule is human serum albumin.

42. The method of claim 39, wherein one or more ligand groups comprise a cyclic RGD peptide attached to a polyethylene glycol (PEG) group through a maleimide group, and the PEG group is attached to the protein.

43. The method of claim 42, wherein the PEG group is attached to the protein through an amide linkage.

44. The method of claim 32, wherein the one or more oligonucleotide groups are attached to the carrier macromolecule through a —(CH₂)₆-alkylene linker group.

45-50. (canceled)
